(19) United States
(12) Patent Application Publication
(10) Pub. No.: US 2022/0135650 A1
(43) Pub. Date: May 5, 2022

(54) ENGINEERED CD47 EXTRACELLULAR DOMAIN FOR BIOCONJUGATION

(71) Applicant: The Board of Trustees of the Leland Stanford Junior University, Stanford, CA (US)

(72) Inventors: James R. Swartz, Menlo Park, CA (US); Maya Nagasawa, Menlo Park, CA (US)

(21) Appl. No.: 17/435,325

(22) PCT Filed: Mar. 2, 2020
(86) PCT No.: PCT/US2020/020671

§ 371 Date: Aug. 31, 2021

Related U.S. Application Data
(60) Provisional application No. 62/813,510, filed on Mar. 4, 2019.

(51) Int. Cl. C07K 14/705; A61L 31/10

(52) U.S. Cl. CPC C07K 14/70596; A61K 38/00; A61L 31/10

(57) ABSTRACT

Compositions and methods are provided relating to engineered CD47 extracellular domain (ECD) proteins.

### CROSS REFERENCE

This application claims benefit of U.S. Provisional Patent Application No. 62/813,510, filed Mar. 4, 2019, which applications are incorporated herein by reference in their entirety.

Nanoparticle (NP)-based targeted delivery is a promising approach for drug delivery, including improved therapy and diagnosis for cancer. However, there are challenges to be overcome for effective delivery of cargo. In addition to particle stability and specific targeting, immune system avoidance is a significant challenge. Only a small percentage of the injected NP dose reaches targeted tissues because the majority of administered NPs suffer are cleared by the mononuclear phagocytic system. The liver and spleen are involved, and for smaller NP the renal system is also involved in clearance. Coating nanoparticles with polyethylene glycol (PEG) can help avoid phagocytes including Kupffer cells in the liver and extend the blood circulation time by creating “stealth” brushes (see Hong et al. Clin. Cancer Res. 5, 3645-52 (1999); and Armstrong et al. Cancer 110, 103-11 (2007)). However, PEGylation can reduce NP uptake by the targeted cells and is potentially immunogenic. Also, such NPs can be opsonized by serum proteins such as IgGs overtime, which increases clearance by phagocytic cells (see Rodriguez et al. Science 339, 971-5 (2013)).

CD47 is a broadly expressed transmembrane glycoprotein with a single Ig-like extracellular domain and five membrane spanning regions. It functions as a cellular ligand for SIRPα with binding mediated through the NH₂-terminal V-like domain of SIRPα. SIRPα is expressed primarily on myeloid cells, including macrophages, granulocytes, myeloid dendritic cells (DCs), mast cells, and their precursors, including monocytes and hematopoietic stem cells. Structural determinants on SIRPα that mediate CD47 binding are discussed by Lee et al. (2007) J. Immunol. 179:7741-7750; Hatherley et al. (2007) J.B.C. 282:14567-75; and the role of SIRPα cis dimerization in CD47 binding is discussed by Lee et al. (2010) J.B.C. 285:37953-63 and reviewed by Barclay and van den Berg Annu. Rev. Immunol. 32, 25-50 (2014))

The extracellular domain (ECD) of CD47 has been proposed as a means of avoiding phagocytosis. When the CD47 ECD interacts with a SIRPα ECD displayed on the surface of phagocytes, it sends a “don't eat-me” signal to inhibit phagocytosis. Foreign materials such as bacteria, viruses and NPs are engulfed by phagocytes, in part because they lack surface CD47. Polymer-based NPs and P22 virus-like particles (VLPs) displaying the CD47 ECD or the CD47 ‘self’ peptide on their surfaces have been reported to have less phagocytic clearance (see Qie et al. Sci. Rep. 6, 26269 (2016) and Schwarz et al. ACS Nano 9, 9134-47 (2015)).

Improved methods to reduce undesirable phagocytic clearance are of interest for development of implantable device and drug delivery modalities. These are addressed herein.

### SUMMARY

Compositions and methods are provided relating to engineered CD47 extracellular domain (ECD) proteins. The CD47 ECD is modified by specific amino acid changes to provide for utility in conjugation to surfaces, where the CD47-ECD is properly oriented on the surface and modified to engage with its counter-receptor, SIRPα. The engagement with SIRPα is required for the biological activity of CD47-ECD in reducing phagocytic clearance. Modifications for these purposes include, without limitation, (i) addition of a cleavable N-terminal extension to produce a pyroglutamate N-terminus; and (ii) substitution of residues to allow introduction of non-natural amino acids (nnAA) at desired attachment sites of the CD47-ECD to the surface. The CD47-ECD can be produced using cell free protein synthesis (CFPS). Reference may be made to the human CD47-ECD, but similar changes have also been made to the mouse CD47-ECD.

In some embodiments, a cleavable extension is provided at the N-terminus of the ECD, where the cleavage site for the extension is immediately adjacent to the N-terminal glutamine of the mature CD47-ECD. Upon cleavage of the extension, this glutamine is exposed at the N-terminus. In some embodiments the exposed glutamine is converted to pyroglutamate with glutaminyl cyclase. In some embodiments the extension comprises a recognition site for enterokinase, e.g. DDDDK, and is cleaved by enterokinase. In some embodiments the extension comprises a recognition site for Factor Xa cleavage, e.g. IEGR and is cleaved by Factor Xa. The extension optionally comprises a tag for purification, e.g. a histidine tag, etc., and may comprise a short linker between the tag and the cleavage site.

At the desired sites for attachment of the CD47-ECD to a surface, nnAA are introduced. The sites for introduction of the nnAA may be the naturally occurring double linkage sites, C15 and V116 (relative to the reference human CD47 ECD of SEQ ID NO:1). Non-natural amino acids for this purpose are selected to provide a reactant group for Click chemistry or for other bioorthogonal reactions. An nnAA may comprise, for example, an alkyne or azide functional group, e.g. homopropargylglycine (HPG) or azidohomoalanine (AHA), respectively. Conveniently this is accomplished by global methionine replacement, although other methods of introducing nnAA may alternatively be used. In embodiments where the nnAA are introduced by methionine replacement, both the C15 and V116 codons in the coding sequence are changed to ATG.

In those embodiments utilizing methionine replacement, the CD47-ECD may also be engineered to replace naturally occurring methionines at sites that are not desirable sites for attachment, i.e. at M28 and M82 (numbering relative to the human reference protein). In some embodiments M28 and M82 are substituted with an amino acid other than methionine. In some embodiments the substituting amino acid is a conservative mutation, e.g. a hydrophobic amino acid such as L, I, V, F, etc. In some embodiments the specific amino acid substitutions are M28V; and M82L/I.

Optionally, the CD47-ECD is further engineered to improve protein solubility. For example, hydrophobic residues on the surface of CD47 ECD that in the native protein faces the cell membrane may be replaced with hydrophilic residues. In some embodiments, residues F14 and V115 are replaced with hydrophilic, non-charged amino acids. In some embodiments the amino acid substitutions are one or both of F14N and V115N.

In an embodiment, an engineered CD47-ECD provided, comprising changes relative to the native protein of (i) addition of a cleavable N-terminal extension to produce a pyroglutamate N-terminus; and (ii) substitution of residues to allow introduction of non-natural amino acids (nnAA) at desired attachment sites of the CD47-ECD to the surface. In some embodiments nnAA are introduced as positions C15 and V116. In some embodiments the nnAA comprise an alkyne or azide functional group. In some embodiments the engineered protein further comprises change (iii) replacement of naturally occurring methionines at M28 and M82. In some embodiments the substituting amino acid is a conservative mutation. In some embodiments the engineered CD47-ECD further comprises change (iv) replacement of hydrophobic residues on the membrane facing surface. In some embodiments residues F14 and V115 are replaced with hydrophilic, non-charged amino acids.

Methods are provided for the use of an engineered CD47-ECD as described herein to coat the surface of an article, usually an article intended for internal use or administration that will be exposed to phagocytic cells, e.g. particles for internal delivery of therapeutic agents such as nanoparticles; microparticles; inserts; sustained release implants, which may be biodegradable; osmotic pumps; implanted devices and prosthetics; and the like. In some embodiments the coating with CD47-ECD reduces phagocytic clearance of the article.

Methods of coating take advantage of the reactive groups in the nnAA to provide a linkage, e.g. a covalent linkage, between the CD47-ECD and reactive groups provided on the surface. The surface reactant groups provide for spacing of reactants as an array, or as pairs. Preferable pairs of reactants are from about 5 to about 15 Å apart, from about 7 to about 13 Å apart, and may be around 10 Å apart. Alternatively an array of reactants is provided on the surface providing a plurality of reactants; or an array that displays the reactive groups on linkers that are fixed to a surface with inconsistent spacing but have the freedom to bring the reactive groups to a spacing that allows the two point attachment. Unreacted groups may be blocked after the CD47-ECD is joined.

In one such embodiment, nnAA at positions 15 and 116 of the CD47-ECD provides either alkyne, or azide functional groups. The surface provides the reactant for the nnAA, i.e. azide to alkyne, or alkyne to azide. Linkage is accomplished by copper(I)-catalyzed azide-alkyne cycloaddition (the “click” reaction). Cleavage of the N-terminal extension and conversion of glutamine to pyroglutamate may be performed before or after the click reaction.

In some embodiments the article is a nanoparticle. In some embodiments the article, including without limitation nanoparticles, comprises proteins that provide the reactant group for linkage to the CD47-ECD. In some embodiments the reactant group on the protein is a nnAA. In some embodiments the nanoparticle is a virus-like particle, and the reactant protein is a virus core protein. In some embodiments the protein is hepatitis B core protein.

In other embodiments, an article is provided, the article comprising engineered CD47-ECD as described herein on the surface. Article include, for example, particles for internal delivery of therapeutic agents such as nanoparticles; microparticles; inserts; sustained release implants, which may be biodegradable; osmotic pumps; implanted devices and prosthetics; and the like. In some embodiments the coating with CD47-ECD reduces phagocytic clearance of the article relative to an article in the absence of the engineered CD47-ECD. In such embodiments the optimal number and spacing of the attached engineered CD47-ECD will be determined by experimentation known to those skilled in the art. In some embodiments the CD47-ECD is covalently linked to a protein present in the article, including without limitation proteins present in virus-like particles. The coated articles may be purified and formulated in pharmacologically acceptable vehicles for administration to a patient. In some embodiments the articles are VLPs covalently joined to engineered CD47-ECDs for formulation. In some embodiments the VLP comprises proteins or drugs for delivery.

The engineered CD47-ECD can be made by generating a nucleic acid construct encoding the engineered protein and producing the polypeptide by cell free synthesis, which synthesis may include coupled transcription and translation reactions. CFPS provides a convenient method for introducing nnAA during synthesis, e.g. using orthogonal tRNAs, global methionine replacement, and the like. Also provided are vectors and polynucleotides encoding the engineered protein.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. CD47 sends a ‘don't eat-me’ survival signal when it interacts with SIRPα on phagocytes.

FIG. 2. Structure of the human CD47 ECD and its binding partner, the human SIRPα domain1. The CD47 ECD's FG loop region (amino acid position 97-106, shown in pink) and the N-terminal (NT) pyroglutamate (shown in light green) are important for binding to SIRPα. The ECD is anchored to the cell surface by its C-terminal (CT) polypeptide and the disulfide bond link at the C15 position.

FIG. 3: Introducing an N-terminal extension to the human CD47 ECD to expose the pyroglutamic acid on the N-terminus after enzymatic reactions. The fusion protein was first digested with the enterokinase to remove the N-terminal tag. Then, the exposed N-terminal glutamine was converted to pyroglutamate using glutaminyl cyclase.

FIG. 4A-4C. Introducing mutations to the CD47 ECD for conjugation to the VLP surface and improved production. (FIG. 4A) Sites at which mutations are introduced to the human CD47 ECD for nnAA incorporation by global methionine replacement. (FIG. 4B) Creating a double linkage between the CD47 ECD and the HepBc dimer to mimic the natural anchoring of the CD47 ECD to the cell membrane. (FIG. 4C) Replacing hydrophobic amino acids to improve protein solubility. Original methionine positions to be replaced to avoid nnAA incorporation at these sites are shown in green, and nnAA incorporation sites for VLP attachment are shown in yellow. The hydrophilic mutation sites are shown in blue.

FIG. 5: The CD47 ECD amino acid sequence conservation.

FIG. 6A-6B: Total cell-free accumulation level and soluble accumulation of the human CD47 ECD mutants. (FIG. 6A) Total product and soluble product accumulation, and (FIG. 6B) protein solubility of the human CD47 ECD mutants expressed with methionine, HPG, or AHA. WT C15G has only the C15G mutation. nnAA(L/I) mutants have C15M, M28V, M82LorI, and V116M. nnAA(L/I) NN mutants have F14N and V115N in addition to the nnAA(L/I) mutations. In this notation, C15M and V116M refer to changes in the coding region that change the native codon to ATG.

FIG. 7A-7B: Purification and production of the human CD47 ECD with pyroglutamate at the N-terminus. (FIG. 7A) SDS-PAGE gel of the human CD47 ECD nnAA(I) NN samples at each purification step. Lane 1: protein ladder (Mark 12), Lane2: after CFPS and buffer exchange, Lane 3: after aggregate removal, Lane 4: Ni-NTA elution, Lane 5: after the enterokinase reaction, Lane 6: purified (1Q)hCD47 ECD nnAA(I) NN. (FIG. 7B) Detection of pyroglutamate formation by QC as indicated by NADH consumption. GLDH=Glutamate dehydrogenase; QC=glutaminyl cyclase.

FIG. 8: Comparison of the human CD47 ECD and the mouse CD47 ECD mutants. Shown is the amino acid sequence when methionine is incorporated instead of nnAA.

FIG. 9A-9B: Cell-free protein total accumulation and soluble protein accumulation for the mouse CD47 ECD mutant. (FIG. 9A) Cell-free protein expression level and (FIG. 9B) protein solubility of the mouse CD47 ECD nnAA(I).

FIG. 10A-10C: Mouse CD47 ECD attachment to the HepBc VLPs. (FIG. 10A) Reducing SDS-PAGE and autoradiogram of the mouse CD47 (mCD47) ECD-attached and disassembled VLP. (Only the HepBc protein is radioactive.) (FIG. 10B) mCD47 ECD number attached to the VLP surface. (FIG. 10C) Surface attachment sites on the HepBc dimer. (For clarity, only the sites in one of the monomers are indicated. Sites on the back side of the molecule are indicated in parentheses.) 79nnAA and 80nnAA refer to the VLPs with the nnAAs incorporated at these sites at the tip of the VLP surface spikes.

FIG. 11: Fluorescent images of RAW 264.7 cell internalization of BDFL-loaded VLPs. The BDFL dyes in the HepBc VLPs are shown in green, and the cell nuclei are shown in magenta. The uptake of the BDFL-loaded VLPs by RAW 264.7 cells was detected by the BDFL fluorescence (middle). Compared to this, the uptake of the mouse CD47 ECD-functionalized BDFL-VLPs was significantly reduced (right).

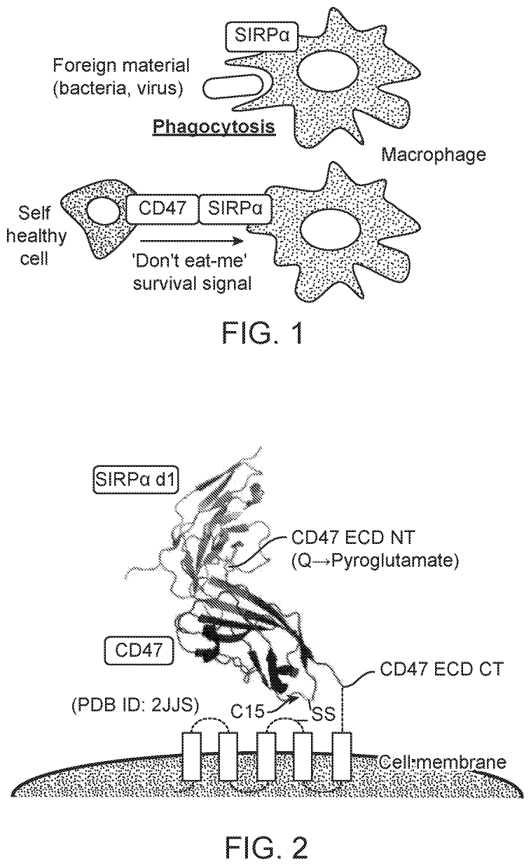

### DETAILED DESCRIPTION OF THE EMBODIMENTS

Compositions and methods are provided relating to engineered CD47 extracellular domain (ECD) proteins. The CD47 ECD is modified by specific amino acid changes to provide for utility in conjugation to surfaces, where the CD47-ECD is properly oriented on the surface and modified to engage with its counter-receptor, SIRPα. The engagement with SIRPα is required for the biological activity of CD47-ECD in reducing phagocytic clearance. Modifications for these purposes include, without limitation, (i) addition of a cleavable N-terminal extension to produce a pyroglutamate N-terminus; and (ii) substitution of residues to allow introduction of non-natural amino acids (nnAA) at desired attachment sites of the CD47-ECD to the surface.

In some embodiments, provided is a use of an engineered protein or an article comprising an engineered protein disclosed herein, for example in the manufacture of a medicament. In an embodiment, provided is a use of an engineered protein or an article comprising an engineered protein disclosed herein for delivery of an agent in the article to a tissue.

CD47 is a 50 kDa transmembrane receptor that has extracellular N-terminal IgV domain, five transmembrane domains, and a short C-terminal intracellular tail. There are four alternatively spliced isoforms of CD47 that differ only in the length of their cytoplasmic tail. It binds to signal-regulatory protein alpha (SIRPα). The CD47/SIRPα interaction leads to bidirectional signaling, resulting in different cell-to-cell responses including inhibition of phagocytosis, stimulation of cell-cell fusion, and T-cell activation, and leads to its activity as a don't eat me signal to phagocytic cells of the immune system. For example, red blood cells that lack CD47 are rapidly cleared from the bloodstream by macrophages, a process that is mediated by interaction with SIRPα.

Sequences of human and mouse CD47 are publicly available, for example the human protein reference sequence at Genbank is NP_034711.1 and the mouse reference protein sequence is NP_001768.1. For convenience, numbering of amino acid residue may be made herein with reference to SEQ ID NO:1, human CD47-ECD QLLFNKTKSVEFTFCNDTWIPCFVTNMEAQNTTEVYVKWKFKGRDIYTFDGALNKSTVPTDFS SAKIEVSQLLKGDASLKMDKSDAVSHTGNYTCEVTELTREGETIIELKYRWS and to the mouse CD47-ECD, SEQ ID NO:2 QLLFSNVNSIEFTSCNETWIPCIVRNVEAQSTEEMFVKWKLNKSYIFIYDGNKNSTTTDQNFTS AKISVSDLINGIASLKMDKRDAMVGNYTCEVTELSREGKTVIELKNRTS

For physiologically relevant purposes the binding of SIRPα and CD47 is usually an event between SIRPα on phagocytic cells and their precursors (e.g., macrophages and monocytes); and CD47 on articles, particularly particulate articles such as nanoparticles, microparticles, etc. that can be targets for phagocytosis.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymers.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refers to compounds that have the same basic chemical structure as a naturally occurring amino acid, i.e., an .alpha. carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Engineered polypeptides disclosed herein may comprise at least one unnatural amino acid at a pre-determined site, and may comprise or contain 1, 2, 3, 4, 5 or more unnatural amino acids. If present at two or more sites in the polypeptide, the unnatural amino acids can be the same or different. Where the unnatural amino acids are different, an orthogonal tRNA and cognate tRNA synthetase will be present for each unnatural amino acid. In some embodiments a single unnatural amino acid is present at residue 80.

Examples of unnatural amino acids that can be used include: an unnatural analogue of a tyrosine amino acid; an unnatural analogue of a glutamine amino acid; an unnatural analogue of a phenylalanine amino acid; an unnatural analogue of a methionine amino acid; an unnatural analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or any combination thereof; an amino acid with a photoactivatable cross-linker; a spin-labeled amino acid; a fluorescent amino acid; an amino acid with a novel functional group; an amino acid that covalently or noncovalently interacts with another molecule; a metal binding amino acid; a metal-containing amino acid; a radioactive amino acid; a photocaged and/or photoisomerizable amino acid; a biotin or biotin-analogue containing amino acid; a glycosylated or carbohydrate modified amino acid; a keto containing amino acid; amino acids comprising polyethylene glycol or polyether; a heavy atom substituted amino acid; a chemically cleavable or photocleavable amino acid; an amino acid with an elongated side chain; an amino acid containing a toxic group; a sugar substituted amino acid, e.g., a sugar substituted serine or the like; a carbon-linked sugar-containing amino acid; a redox-active amino acid; an α-hydroxy containing acid; an amino thio acid containing amino acid; an α,α disubstituted amino acid; a β-amino acid; a cyclic amino acid other than proline, etc.

Unnatural amino acids of interest include, without limitation, amino acids that provide a reactant group for CLICK chemistry reactions (see Click Chemistry: Diverse Chemical Function from a Few Good Reactions Hartmuth C. Kolb, M. G. Finn, K. Barry Sharpless Angewandte Chemie International Edition Volume 40, 2001, P. 2004, herein specifically incorporated by reference). For example, the amino acids azidohomoalanine, homopropargyiglycine, p-acetyl-L-phenylalanine and p-azido-L-phenylalanine are of interest.

In some embodiments, the unnatural amino acid is introduced by global replacement of methionine on the protein, e.g. methionine can be left out of a cell-free reaction mixture, and substituted by from 0.25-2.5 mM azidohomoalanine (AHA). In such embodiments it is preferred to substitute natural methionines with a different amino acid, while an ATG codon is introduced into the coding sequence at the desired site for unnatural amino acid introduction.

Alternatively the unnatural amino acid is introduced by orthogonal components. Orthogonal components include a tRNA aminoacylated with an unnatural amino acid, where the orthogonal tRNA base pairs with a codon that is not normally associated with an amino acid, e.g. a stop codon; a 4 bp codon, etc. The reaction mixture may further comprise a tRNA synthetase capable of aminoacylating (with an unnatural amino acid) the cognate orthogonal tRNA. Such components are known in the art, for example as described in U.S. Pat. No. 7,045,337, issued May 16, 2006. The orthogonal tRNA recognizes a selector codon, which may be nonsense codons, such as, stop codons, e.g., amber, ochre, and opal codons; four or more base codons; codons derived from natural or unnatural base pairs and the like. The orthogonal tRNA anticodon loop recognizes the selector codon on the mRNA and incorporates the unnatural amino acid at this site in the polypeptide.

Orthogonal tRNA synthetase can be synthesized exogenously, purified and added to the reaction mix of the invention, usually in a defined quantity, of at least about 10 μg/ml, at least about 20 μg/ml, at least about 30 μg/ml, and not more than about 200 μg/ml. The protein may be synthesized in bacterial or eukaryotic cells and purified, e.g. by affinity chromatography, PAGE, gel exclusion chromatography, reverse phase chromatography, and the like, as known in the art.

The terms “conjugation partner” may be used to refer to any moiety, for example a peptide or protein, nucleic acid, polysaccharide, label, etc. that is conjugated to the engineered CD47-ECD. The conjugation partner may be present on the surface of an article, or may form the surface of an article. The conjugation partner may comprise a complementary active group for CLICK chemistry conjugation, for example the conjugation partner may be synthesized with one or more unnatural amino acids, which allow for the conjugation to the unnatural amino acid present on the CD47-ECD protein. One of skill in the art will understand that the chemistry for conjugation is well-known and can be readily applied to a variety of groups, e.g. CpG DNA sequences, detectable label, antigen, polypeptide, etc.

In some embodiments the conjugation partner is a structural protein, e.g. a collagen, keratin, actin, myosin, elastin, fibrillin, lamin, etc. In some embodiments the conjugation partner is a protein of a virus like particle, including without limitation, hepatitis B core protein HBc. In some embodiments the conjugation partner is a polymer, e.g. PEG, etc., which has been modified to provide reactive groups.

The term “HBc” refers to the amino acid peptide sequence of the Hepatitis B core protein, or to a truncated version thereof, or a comparable protein, for example a protein modified with one or more disulfide bonds; modified to provide a site for introduction of an non-natural amino acid, comprising tip modifications and the like as set forth in U.S. Pat. No. 9,896,483, herein specifically incorporated by reference. In some embodiments HBc is a conjugation partner for engineered CD47-ECD.

Various groups may be introduced into the peptide during synthesis or during expression, which allow for linking to molecules such as CD47-ECD, or to a surface. Further such surfaces, if modified to display reactive species, may first be modified by linking articles, for example VLPs, to said surface followed by linkage of engineered CD47-ECDs to the articles. For example, the surface may first display alkynes allowing linkage of VLPs displaying azides, followed by linkage of engineered CD47-ECDs displaying alkynes. The introduced groups need not be included in the HBc domain itself, but may be introduced as a tag or fusion C-terminal or N-terminal to the HBc domain. Thus cysteines can be used to make thioethers, poly histidines for linking to a metal ion complex, carboxyl groups for forming amides or esters, amino groups for forming amides, and the like. An insertion of 3 amino acids (ASV) after the initiator formyl-methionine to remove a translation initiating non-natural methionine analog by methionyl aminopeptidase may be included to avoid surface conjugation at undesired positions.

In some embodiments an unnatural amino acid is included at one or more defined sites in the protein. The HBc polypeptides of the invention may include an unnatural amino acid for the control of direct attachment to the conjugation partner CD47-ECD. The nnAA on the conjugation partner is different from, and reactive with, the nnAA present on the HBc polypeptide(s).

One of skill in the art will understand that minor amino acid changes can be made in the sequence without altering the function of the protein, e.g. changes of 1, 2, 3, 4, 5, 6, 7, 8, 9, or up to about 10 amino acids, and that a full-length protein may be substituted for the truncated versions exemplified herein. HBc is functionally capable of self-assembling to form an icosahedral virus like particle. The HBc polypeptides of the invention may also comprise a cargo-loading domain.

As used herein, the term “virus like particle” refers to a stable macromolecular assembly of one or more virus proteins, usually viral coat proteins. The number of separate protein chains in a VLP will usually be at least about 60 proteins, about 80 proteins, at least about 120 proteins, or more, depending on the specific viral geometry. In the methods of the invention, the VLP comprises HBc conjugated to an engineered CD47-ECD. The methods of the invention provide for synthesis of the coat protein in the absence of the virus polynucleotide genome, and thus the capsid may be empty, or contain non-viral components, e.g. mRNA fragments, drug cargo, etc.

A stable VLP maintains the association of proteins in a capsid structure under physiological conditions for extended periods of time, e.g. for at least about 24 hrs, at least about 1 week, at least about 1 month, or more. Once assembled, the VLP can have a stability commensurate with the native virus particle, e.g. upon exposure to pH changes, heat, freezing, ionic changes, etc. Additional components of VLPs, as known in the art, can be included within or disposed on the VLP. VLPs do not contain intact viral nucleic acids, and they are non-infectious. In some embodiments there is sufficient viral surface envelope glycoprotein and/or adjuvant molecules on the surface of the VLP so that when a VLP preparation is formulated into an immunogenic composition and administered to an animal or human, an immune response (cell-mediated or humoral) is raised.

As used herein, the terms “purified” and “isolated” when used in the context of a polypeptide that is substantially free of contaminating materials from the material from which it was obtained, e.g. cellular materials, such as but not limited to cell debris, cell wall materials, membranes, organelles, the bulk of the nucleic acids, carbohydrates, proteins, and/or lipids present in cells. Thus, a polypeptide that is isolated includes preparations of a polypeptide having less than about 30%, 20%, 10%, 5%, 2%, or 1% (by dry weight) of cellular materials and/or contaminating materials. As used herein, the terms “purified” and “isolated” when used in the context of a polypeptide that is chemically synthesized refers to a polypeptide which is substantially free of chemical precursors or other chemicals which are involved in the syntheses of the polypeptide.

The polypeptides may be isolated and purified in accordance with conventional methods of recombinant synthesis or cell free protein synthesis. Separation procedures of interest include affinity chromatography. Affinity chromatography makes use of the highly specific binding sites usually present in biological macromolecules, separating molecules on their ability to bind a particular ligand. Covalent bonds attach the ligand to an insoluble, porous support medium in a manner that overtly presents the ligand to the protein sample, thereby using natural biospecific binding of one molecular species to separate and purify a second species from a mixture. Antibodies are commonly used in affinity chromatography. Preferably a microsphere or matrix is used as the support for affinity chromatography. Such supports are known in the art and are commercially available, and include activated supports that can be combined to the linker molecules. For example, Affi-Gel supports, based on agarose or polyacrylamide are low pressure gels suitable for most laboratory-scale purifications with a peristaltic pump or gravity flow elution. Affi-Prep supports, based on a pressure-stable macroporous polymer, are suitable for preparative and process scale applications.

Proteins may also be separated by ion exchange chromatography, and/or concentrated, filtered, dialyzed, etc., using methods known in the art. The methods of the present invention provide for proteins containing unnatural amino acids that have biological activity comparable to the native protein. One may determine the specific activity of a protein in a composition by determining the level of activity in a functional assay, quantitating the amount of protein present in a non-functional assay, e.g. immunostaining, ELISA, quantitation on coomassie or silver stained gel, etc., and determining the ratio of biologically active protein to total protein. Generally, the specific activity as thus defined will be at least about 5% that of the native protein, usually at least about 10% that of the native protein, and may be about 25%, about 50%, about 90% or greater.

Exemplary coding sequences are provided, however one of skill in the art can readily design a suitable coding sequence based on the provided amino acid sequences. Methods which are well known to those skilled in the art can be used to construct expression vectors containing coding sequences and appropriate transcriptional/translational control signals. These methods include, for example, in vitro recombinant DNA techniques, synthetic techniques and in vivo recombination/genetic recombination. Alternatively, RNA capable of encoding the polypeptides of interest may be chemically synthesized. One of skill in the art can readily utilize well-known codon usage tables and synthetic methods to provide a suitable coding sequence for any of the polypeptides of the invention. The nucleic acids may be isolated and obtained in substantial purity. Usually, the nucleic acids, either as DNA or RNA, will be obtained substantially free of other naturally-occurring nucleic acid sequences, generally being at least about 50%, usually at least about 90% pure and are typically “recombinant,” e.g., flanked by one or more nucleotides with which it is not normally associated on a naturally occurring chromosome. The nucleic acids of the invention can be provided as a linear molecule or within a circular molecule, and can be provided within autonomously replicating molecules (vectors) or within molecules without replication sequences. Expression of the nucleic acids can be regulated by their own or by other regulatory sequences known in the art. The nucleic acids of the invention can be introduced into suitable host cells using a variety of techniques available in the art.

The term “diagnosis” is used herein to refer to the identification of a molecular or pathological state, disease or condition. The term “prognosis” is used herein to refer to the prediction of the likelihood of cancer-attributable death or progression, including recurrence, metastatic spread, and drug resistance. The term “prediction” is used herein to refer to the act of foretelling or estimating, based on observation, experience, or scientific reasoning. In one example, a physician may predict the likelihood that a patient will survive, following a treatment.

As used herein, the terms “treatment,” “treating,” and the like, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease.

Treating may refer to any indicia of success in the treatment or amelioration or prevention of disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with cancer or other diseases. The term “therapeutic effect” refers to the reduction, elimination, or prevention of the disease, symptoms of the disease, or side effects of the disease in the subject.

“In combination with”, “combination therapy” and “combination products” refer, in certain embodiments, to the concurrent administration to a patient of a first therapeutic and the compounds as used herein. When administered in combination, each component can be administered at the same time or sequentially in any order at different points in time. Thus, each component can be administered separately but sufficiently closely in time so as to provide the desired therapeutic effect.

An “effective amount” or a “sufficient amount” of a substance is that amount sufficient to cause a desired biological effect, such as beneficial results, including clinical results, and, as such, an “effective amount” depends upon the context in which it is being applied. An effective amount can be administered in one or more administrations. For example an effective amount or density of engineered CD47-ECD in a particle may be that amount or density that reduces phagocytic clearance by at least 10%, at least 20%, at least 50%, at least 75%, at least 90%, at least 95% or more, relative to a particle that lacks the CD47-ECD.

Cargo. The VLP may encapsulate cargo, e.g. a molecule that will be released when the VLP is inside a cell. Encapsulated cargo is protected within the VLP, and is typically not displayed on the surface of the VLP. Stably loaded cargo is retained within a VLP after washing.

Many molecules are suitable as cargo, including without limitation RNA, e.g. guide RNA, siRNA, antisense RNA and the like; DNA, e.g. double stranded or single stranded DNA, including without limitation plasmids, coding sequences, etc.; proteins such as toxin proteins including, for example, diphtheria A chain, exotoxin A chain, ricin A chain, abrin A chain, curcin, crotin, phenomycin, enomycin, auristatin-E and the like; genetic modifying proteins including without limitation CRISPR; binding proteins such as antibodies or fragments derived therefrom, and the like. Cytotoxic agents are numerous and varied. One exemplary class of cytotoxic agents are chemotherapeutic agents. Exemplary chemotherapeutic agents include, but are not limited to, aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, duocarmycin, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, mertansine, metoclopramide, mitomycin, mitotane, mitoxantrone, omeprazole, ondansetron, paclitaxel, pilocarpine, prochloroperazine, rituximab, saproin, tamoxifen, taxol, topotecan hydrochloride, trastuzumab, vinblastine, vincristine, vinorelbine tartrate, etc.

In some embodiments a cargo is modified to enhance encapsulation by the VLP, for example by addition of a free sulfhydryl group for conjugation to the HBc protein. Alternatively a cargo agent may be selected that comprises a free sulfhydryl group, such as mertansine. A polar cargo, e.g. a nucleic acid such as ssDNA, RNA, etc., may be conjugated to a hydrophobic group such as, for example, cholesterol to enhance loading. Alternatively one or more polar or charged amino acids can be conjugated to the cargo.

Cell free protein synthesis, as used herein, refers to the cell-free synthesis of polypeptides in a reaction mix comprising biological extracts and/or defined reagents. The reaction mix will comprise a template for production of the macromolecule, e.g. DNA, mRNA, etc.; monomers for the macromolecule to be synthesized, e.g. amino acids, nucleotides, etc., and such co-factors, enzymes and other reagents that are necessary for the synthesis, e.g. ribosomes, tRNA, polymerases, transcriptional factors, etc. Such synthetic reaction systems are well-known in the art, and have been described in the literature. The cell free synthesis reaction may be performed as batch, continuous flow, or semi-continuous flow, as known in the art.

The CFPS and other subsequent steps may be performed under reducing conditions, e.g. in the presence of 1 mM DTT or the equivalent. Following assembly of the VLP the conditions may be changed to an oxidizing environment, e.g. by dialysis to remove the reducing agent, optionally in the presence of a salt, e.g. up to about 1M salt, up to about 1.5M salt, up to about 2.5 M salt, e.g. NaCl, etc., then oxidizing to form disulfide bonds by adding 5-10 mM H₂O₂, 5-20 mM diamide, or the equivalent.

In some embodiments of the invention, cell free synthesis is performed in a reaction where oxidative phosphorylation is activated, e.g. the CYTOMIM™ system. The activation of the respiratory chain and oxidative phosphorylation is evidenced by an increase of polypeptide synthesis in the presence of O₂. In reactions where oxidative phosphorylation is activated, the overall polypeptide synthesis in presence of O₂ is reduced by at least about 40% in the presence of a specific electron transport chain inhibitor, such as HQNO, or in the absence of O₂. The reaction chemistry may be as described in international patent application WO 2004/016778, herein incorporated by reference.

The CYTOMIM™ environment for synthesis utilizes cell extracts derived from bacterial cells grown in medium containing glucose and phosphate, where the glucose is present initially at a concentration of at least about 0.25% (weight/volume), more usually at least about 1%; and usually not more than about 4%, more usually not more than about 2%. An example of such media is 2YTPG medium, however one of skill in the art will appreciate that many culture media can be adapted for this purpose, as there are many published media suitable for the growth of bacteria such as E. coli, using both defined and undefined sources of nutrients (see Sambrook, J., E. F. Fritsch, and T. Maniatis. 1989. Molecular Cloning: A Laboratory Manual, 2^{nd} edition. Cold Spring Harbor University Press, Cold Spring Harbor, N.Y. for examples of glucose containing media). Alternatively, the culture may be grown using a protocol in which the glucose is continually fed as required to maintain a high growth rate in either a defined or complex growth medium. The reaction mixture may be supplemented by the inclusion of vesicles, e.g. an inner membrane vesicle solution. Where provided, such vesicles may comprise from about 0 to about 0.5 volumes, usually from about 0.1 to about 0.4 volumes.

In some embodiments, PEG will be present in not more than trace amounts, for example less than 0.1%, and may be less than 0.01%. Reactions that are substantially free of PEG contain sufficiently low levels of PEG that, for example, oxidative phosphorylation is not PEG-inhibited. The molecules spermidine and putrescine may be used in the place of PEG. Spermine or spermidine is present at a concentration of at least about 0.5 mM, usually at least about 1 mM, preferably about 1.5 mM, and not more than about 2.5 mM. Putrescine is present at a concentration of at least about 0.5 mM, preferably at least about 1 mM, preferably about 1.5 mM, and not more than about 2.5 mM. The spermidine and/or putrescine may be present in the initial cell extract or may be separately added.

The concentration of magnesium in the reaction mixture affects the overall synthesis. Often there is magnesium present in the cell extracts, which may then be adjusted with additional magnesium to optimize the concentration. Sources of magnesium salts useful in such methods are known in the art. In one embodiment of the invention, the source of magnesium is magnesium glutamate. A preferred concentration of magnesium is at least about 5 mM, usually at least about 10 mM, and preferably a least about 12 mM; and at a concentration of not more than about 25 mM, usually not more than about 20 mM. Other changes that may enhance synthesis or reduce cost include the omission of HEPES buffer and phosphoenol pyruvate from the reaction mixture.

The system can be run under aerobic and anaerobic conditions. Oxygen may be supplied, particularly for reactions larger than 15 μl, in order to increase synthesis yields. The headspace of the reaction chamber can be filled with oxygen; oxygen may be infused into the reaction mixture; etc. Oxygen can be supplied continuously or the headspace of the reaction chamber can be refilled during the course of protein expression for longer reaction times. Other electron acceptors, such as nitrate, sulfate, or fumarate may also be supplied in conjunction with preparing cell extracts so that the required enzymes are active in the cell extract.

It is not necessary to add exogenous cofactors for activation of oxidative phosphorylation. Compounds such as nicotinamide adenine dinucleotide (NADH), NAD⁺, or acetyl-coenzyme A may be used to supplement protein synthesis yields but are not required. Addition of oxalic acid, a metabolic inhibitor of phosphoenolpyruvate synthetase (Pps), may be beneficial in increasing protein yields, but is not necessary.

The template for cell-free protein synthesis can be either mRNA or DNA, preferably a combined system continuously generates mRNA from a DNA template with a recognizable promoter. Either an endogenous RNA polymerase is used, or an exogenous phage RNA polymerase, typically T7 or SP6, is added directly to the reaction mixture. Alternatively, mRNA can be continually amplified by inserting the message into a template for QB replicase, an RNA dependent RNA polymerase. Purified mRNA is generally stabilized by chemical modification before it is added to the reaction mixture. Nucleases can be removed from extracts to help stabilize mRNA levels. The template can encode for any particular gene of interest.

Other salts, particularly those that are biologically relevant, such as manganese, may also be added. Potassium is generally present at a concentration of at least about 50 mM, and not more than about 250 mM. Ammonium may be present, usually at a concentration of not more than 200 mM, more usually at a concentration of not more than about 100 mM. Usually, the reaction is maintained in the range of about pH 5-10 and a temperature of about 20°-50° C.; more usually, in the range of about pH 6-9 and a temperature of about 25°-40° C. These ranges may be extended for specific conditions of interest.

Metabolic inhibitors to undesirable enzymatic activity may be added to the reaction mixture. Alternatively, enzymes or factors that are responsible for undesirable activity may be removed directly from the extract or the gene encoding the undesirable enzyme may be inactivated or deleted from the chromosome.

As used herein, the terms “subject” or “patient” are used interchangeably to refer to an animal (e.g., birds, reptiles, and mammals). In some embodiments, a subject is a mammal including a non-primate (e.g., a camel, donkey, zebra, cow, pig, horse, goat, sheep, cat, dog, rat, and mouse) and a primate (e.g., a monkey, chimpanzee, and a human). In certain embodiments, a subject is a non-human animal. In some embodiments, a subject is a farm animal or pet. In another embodiment, a subject is a human, e.g. a human infant including premature infants, child, adult, and/or elderly human.

By “fused” or “operably linked” herein is meant that two or more polypeptides are linked together to form a continuous polypeptide chain. A fusion polypeptide (or fusion polynucleotide encoding the fusion polypeptide) can comprise further components as well, including multiple peptides at multiple loops, fusion partners, etc. The precise site at which the fusion is made is not critical; particular sites are well known and may be selected in order to optimize the biological activity, secretion or binding characteristics of the binding partner. The optimal site will be determined by routine experimentation.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g., ethanolamine, diethanolamine, triethanolamine, tromethamine, N methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g., C₁₋₆ alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention may be present in more than one stereoisomeric form, and the naming of such compounds is intended to include all single stereoisomers and all mixtures (whether racemic or otherwise) of such stereoisomers.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

### Proteins

Engineered CD47 extracellular domain (ECD) proteins, e.g. human CD47 ECD proteins, are modified by specific amino acid changes to provide for utility in conjugation to surfaces, where the CD47-ECD is properly oriented on the surface and modified to engage with its counter-receptor, SIRPα. Such modifications include, without limitation, (i) addition of a cleavable N-terminal extension to produce a pyroglutamate N-terminus; and (ii) substitution of residues to allow introduction of non-natural amino acids (nnAA) at desired attachment sites of the CD47-ECD to the surface. In certain embodiments the CD47 ECD comprises a sequence of any of one SEQ ID NO:3, 5, 7, 9, 11 or 13, or a sequence substantially similar to any of one SEQ ID NO:3, 5, 7, 9, 11 or 13, e.g. greater than 99% sequence identity, greater than 95% sequence identity, greater than 90% sequence identity.

A cleavable extension is provided at the N-terminus of the ECD, where a specific cleavage recognition site in the extension is immediately adjacent to the N-terminal glutamine of the mature CD47-ECD. Upon cleavage of the extension, this glutamine is exposed at the N-terminus. The exposed glutamine is converted to pyroglutamate, for example with glutaminyl cyclase. The extension may be any length provided a specific cleavage recognition site is included, usually being at least 3, at least 4, at least 5, at least 6 or more residues and may be considerably longer depending on the desired functionality. Conveniently the extension will be not more than about 50 residues, not more than 40, not more than 30, not more than 20 residues in length. The cleavage recognition site may be a recognition site for enterokinase, e.g. DDDDK, for Factor Xa cleavage, e.g. IEGR, etc. where the cognate enzyme is used to cleave the extension free of the CD47-ECD.

An extension optionally comprises a protein tag for purification or other purposes. The tag may provide for purification. Many such tags are known and used in the art, including for example, e.g. a histidine tag, polyglutamate tag, chitin binding protein (CBP), maltose binding protein (MBP), E-tag, FLAG-tag, S-tag, SBP tag, Strep-tag, calmodulin tag, glutathione-S-transferase (GST), etc. The tag may be an epitope tag, e.g. V5-tag, Myc-tag, HA-tag, Spot-tag, NE-tag, Strep-tag, TC tag, Ty tag, V5 tag, VSV-tag, Xpress tag, etc. AviTag provides for biotinylation by the enzyme BirA.

At the desired sites for attachment of the CD47-ECD to a surface, nnAA are introduced. The sites for introduction of the nnAA may be the naturally occurring double linkage sites, C15 and V116 (relative to the reference human CD47 ECD of SEQ ID NO:1). Non-natural amino acids for this purpose are selected to provide a reactant group for Click chemistry or for other bioorthogonal reactions. An nnAA may comprise, for example, an alkyne or azide functional group, e.g. homopropargylglycine (HPG) or azidohomoalanine (AHA), respectively. Conveniently this is accomplished by global methionine replacement, although orthogonal methods of introducing nnAA may alternatively be used. In embodiments where the nnAA are introduced by methionine replacement, the amino acid substitutions may be C15nnAA and V116nnAA.

In those embodiments utilizing methionine replacement, the CD47-ECD may also be engineered to replace naturally occurring methionines at sites that are not desirable sites for attachment, i.e. at M28 and M82 (numbering relative to the human reference protein). In some embodiments M28 and M82 are substituted with an amino acid other than methionine. In some embodiments the substituting amino acid is a conservative mutation, e.g. a hydrophobic amino acid such as L, I, V, F, etc. In some embodiments the specific amino acid substitutions are M28V; and M82L/I.

Optionally, the CD47-ECD is further engineered to improve protein solubility. For example, hydrophobic residues on the surface of CD47 ECD that in the native protein faces the cell membrane may be replaced with hydrophilic residues. In some embodiments, residues F14 and V115 are replaced with hydrophilic, non-charged amino acids. In some embodiments the amino acid substitutions are one or both of F14N and V115N.

A mouse version of the CD47-ECD is also provided, where for example the naturally occurring methionines at residues M36, M82, M88 are replaced with a conservative substitution. A cleavable extension is provided. nnAA are introduced at C15 and V116.

The engineered CD47-ECD can be made by generating a nucleic acid construct encoding the engineered protein and producing the polypeptide by cell free synthesis, which synthesis may include coupled transcription and translation reactions. CFPS provides a convenient method for introducing nnAA during synthesis, e.g. using orthogonal tRNAs, global methionine replacement, and the like. Also provided are vectors and polynucleotides encoding the engineered protein.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like. In some embodiments an unnatural amino acid is included at one or more defined sites in the protein, particularly at the tip of the protruding surface spikes on the VLP to provide steric availability.

The invention further provides nucleic acids encoding the polypeptides. As will be appreciated by those in the art, due to the degeneracy of the genetic code, an extremely large number of nucleic acids may be made, all of which encode the fusion proteins of the present invention. Thus, having identified a particular amino acid sequence, those skilled in the art could make any number of different nucleic acids, by simply modifying the sequence of one or more codons in a way that does not change the amino acid sequence of the protein.

Using the nucleic acids of the present invention that encode a protein, a variety of expression constructs can be made. The expression constructs may be self-replicating extrachromosomal vectors or vectors which integrate into a host genome. Alternatively, for purposes of cell-free expression, the construct may include those elements required for transcription and translation of the desired polypeptide but may not include such elements as an origin of replication, selectable marker, etc. Cell-free constructs may be replicated in vitro, e.g. by PCR, and may comprise terminal sequences optimized for amplification reactions.

Generally, expression constructs include transcriptional and translational regulatory nucleic acid operably linked to the nucleic acid encoding the fusion protein. The term “control sequences” refers to DNA sequences necessary for the expression of an operably linked coding sequence in a particular expression system, e.g. mammalian cell, bacterial cell, cell-free synthesis, etc. The control sequences that are suitable for prokaryote systems, for example, include a promoter, optionally an operator sequence, and a ribosome binding site. Eukaryotic cell systems may utilize promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate the initiation of translation. Generally, “operably linked” means that the DNA sequences being linked are contiguous, and, in the case of a secretory leader, contiguous and in reading phase. Linking is accomplished by ligation or through amplification reactions. Synthetic oligonucleotide adaptors or linkers may be used for linking sequences in accordance with conventional practice.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention. In a preferred embodiment, the promoters are strong promoters, allowing high expression in in vitro expression systems, such as the T7 promoter.

In addition, the expression construct may comprise additional elements. For example, the expression vector may have one or two replication systems, thus allowing it to be maintained in organisms, for example in mammalian or insect cells for expression and in a prokaryotic host for cloning and amplification. In addition the expression construct may contain a selectable marker gene to allow the selection of transformed host cells. Selection genes are well known in the art and will vary with the host cell used.

### Formulations and Uses

Methods are provided for the use of an engineered CD47-ECD to covalently link to the surface of an article, usually an article intended for internal use or administration that will be exposed to phagocytic cells, e.g. particles for internal delivery of therapeutic agents such as nanoparticles; microparticles; inserts; sustained release implants, which may be biodegradable; osmotic pumps; implanted devices and prosthetics; and the like. In some embodiments the coating with CD47-ECD reduces phagocytic clearance of the article.

Methods of coating take advantage of the reactive groups in the nnAA to provide a linkage, e.g. a covalent linkage, between the CD47-ECD and reactive groups provided on the surface. The surface reactant groups provide for spacing of reactants as an array, or as pairs. Preferable pairs of reactants are from about 5 to about 15 Å apart, from about 7 to about 13 Å apart, and may be around 10 Å apart. Alternatively an array of reactants is provided on the surface providing a plurality of reactants; or an array that displays the reactive groups on linkers that are fixed to a surface with inconsistent spacing but have the freedom to bring the reactive groups to a spacing that allows the two point attachment. Unreacted groups may be blocked after the CD47-ECD is joined.

In one such embodiment, nnAA at positions 15 and 116 of the CD47-ECD provides either alkyne, or azide functional groups. The surface provides the reactant for the nnAA, i.e. azide to alkyne, or alkyne to azide. Linkage is accomplished by copper(I)-catalyzed azide-alkyne cycloaddition (the “click” reaction). Cleavage of the N-terminal extension and conversion of glutamine to pyroglutamate may be performed before or after the click reaction.

In some embodiments the article is a nanoparticle. In some embodiments the article, including without limitation nanoparticles, comprises proteins that provide the reactant group for linkage to the CD47-ECD. In some embodiments the reactant group on the protein is a nnAA. In some embodiments the nanoparticle is a virus-like particle, and the reactant protein is a virus core protein. In some embodiments the protein is hepatitis B core protein.

In other embodiments, an article is provided, the article comprising engineered CD47-ECD as described herein on the surface. Article include, for example, particles for internal delivery of therapeutic agents such as nanoparticles; microparticles; inserts; sustained release implants, which may be biodegradable; osmotic pumps; implanted devices and prosthetics; and the like. In some embodiments the coating with CD47-ECD reduces phagocytic clearance of the article relative to an article in the absence of the engineered CD47-ECD. In some embodiments the CD47-ECD is covalently linked to a protein present in the article, including without limitation proteins present in virus-like particles. The coated articles may be purified and formulated in pharmacologically acceptable vehicles for administration to a patient. In some embodiments the articles are VLPs covalently joined to engineered CD47-ECDs for formulation. In some embodiments the VLP comprises proteins or drugs for delivery.

The compositions of the invention may be formulated as a CD47-ECD suitable for conjugation, e.g. in a kit form to react with a desired surface. Formulations of articles, e.g. nanoparticles, etc. comprising an engineered CD47-ECD are also provided. Such a formulation can be used as a therapeutic or prophylactic, e.g. as a drug delivery vehicle. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LDs/ED₅₀. Compounds that exhibit large therapeutic indices are preferred.

The data obtained from cell culture assays and animal studies can be used in formulating a range of dosage for use in humans. The dosage of such compounds lies preferably within a range of circulating concentrations that includes the ED₅₀ with little or no toxicity. The dosage may vary within this range depending upon the dosage form employed and the route of administration utilized. For any compound used in the method of the invention, the therapeutically effective dose can be estimated initially from cell culture assays.

For all such treatments described above, the exact formulation, route of administration and dosage can be chosen by the individual physician in view of the patient's condition. (See e.g., Fingl et al., 1975, in “The Pharmacological Basis of Therapeutics”). It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administered dose in the management of the viral infection of interest will vary with the severity of the condition to be treated and the route of administration. The dose will also vary according to the age, weight, and response of the individual patient. A program comparable to that discussed above may be used in veterinary medicine.

The pharmacologically active compounds can be processed in accordance with conventional methods of pharmaceutical formulation to produce medicinal agents for administration to patients, e.g., mammals including humans.

Generally, the compositions of the invention preferably also comprise a pharmaceutically acceptable excipient, and may be in various formulations. As is well known in the art, a pharmaceutically acceptable excipient is a relatively inert substance that stabilizes and facilitates administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington's Pharmaceutical Sciences 19th Ed. Mack Publishing (1995).

Generally, these compositions are formulated for administration by injection or inhalation, e.g., intraperitoneally, intravenously, subcutaneously, intradermally, intramuscularly, etc. Accordingly, these compositions may be combined with pharmaceutically acceptable vehicles such as saline, Ringer's solution, dextrose solution, and the like. The particular dosage regimen, i.e., dose, timing and repetition, will depend on the particular individual and that individual's medical history.

It is to be understood that this invention is not limited to the particular methodology, protocols, cell lines, animal species or genera, constructs, and reagents described, as such may, of course, vary. It is also to be understood that the terminology used herein is for the purpose of describing particular embodiments only, and is not intended to limit the scope of the present invention, which will be limited only by the appended claims.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the reagents, cells, constructs, and methodologies that are described in the publications, and which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### Experimental

### Engineering the CD47 Extracellular Domain for Bioconjugation to the Hepatitis B Core Virus-Like Particle Surface to Avoid Phagocyte Engulfment

The CD47 ECD has a potential to be widely used to avoid phagocytes, but the efficient production and purification of functional CD47 ECD using E. coli has been limited. Because of the disulfide bond inside the ECD structure and the natural hydrophobicity, E. coli expression of the CD47 ECD required using a bulky fusion tag such as GST and extensive processing to unfold and re-fold the protein. (see Lin et al. Protein Expr. Purif. 85, 109-116 (2012); and Han et al. J. Biol. Chem. 275, 37984-92 (2000)).

In addition, pyroglutamate formation at the N-terminus, which is important to bind to SIRPα, is a challenge as the N-terminal polypeptide needs to be removed to expose a glutamine residue on the N-terminus, which then needs to be converted to pyroglutamate by glutaminyl cyclase (FIG. 2, see Hatherley et al. Mol. Cell 31, 266-77 (2008)). Although there have been several examples in which the CD47 ECD was expressed in and purified from various kinds of cells including bacteria, mammalian (CHO), and insect (High Five) cells, all of them had an extension on either the N- or C-terminus (see Ho et al. J. Biol. Chem. 290, 12650-63 (2015)).

Mimicking the natural CD47 ECD orientation on the cell surface to achieve the ideal and authentic phagocyte avoidance is another challenge. As shown in Error! Reference source not found., the CD47 ECD is anchored to the cell membrane by a double linkage (polypeptide and disulfide bond) between the ECD and the cell surface.

Here, using E. coli-based cell-free protein synthesis (CFPS), we developed a method to produce the CD47 ECD mutant to be attached on the HepBc VLP surface to avoid immune clearance. First, an N-terminal extension was introduced to help produce the pyroglutamate N-terminus through enzymatic reactions. Second, two non-natural amino acid (nnAA) incorporation sites were introduced so that CD47 ECD attachment to the VLP surface can replicate the natural double linkage between the ECD and the cell membrane. Using copper(I)-catalyzed azide-alkyne cycloaddition (the “click” reaction), two nnAAs on the CD47 ECD can form two covalent linkages with two other nnAAs exposed at the tip of the HepBc VLP surface spike. Then, the CD47 ECD was further engineered to increase its solubility without affecting binding to SIRPα. After this engineered CD47 ECD was expressed and purified, it was attached to the HepBc VLP surface. The CD47 ECD attachment then successfully inhibited VLP engulfment by phagocytes. Combined with recent efforts to engineer HepBc VLPs for targeted delivery of therapeutic cargo (U.S. Patent Application No. 62/785,866), CD47 ECD-functionalized VLPs will greatly increase delivery efficiency and therapeutic efficacy.

### Materials and Methods

Construction of expression plasmids. The original sequence encoding the human CD47 ECD (UniProt accession no. Q08722, mature protein residues 1-117 (original amino acid position 19-135)) was codon optimized for E. coli expression, and synthesized as “gBlocks” from IDT. The gene was cloned into the minimal vector pY71 with restriction enzyme sites NdeI and SalI using Gibson Assembly. Based on this, an N-terminal extension (His₆-EKseq), mutations for non-natural amino acid incorporation (C15M, M28V, M82L/I, V116M), and mutations for improved protein solubility (F14N and V115N) were introduced using QuikChange mutagenesis and primers from IDT. The 5′ untranslated region of the mRNA transcribed from the plasmids was analyzed and optimized to reduce the formation of RNA hairpins using MFold.

A sequence encoding the mouse CD47 ECD (UniProt accession no. Q61735, mature protein residues 1-115 (amino acid position 19-133)) with an N-terminal extension (His₆-EKseq) and mutations for non-natural amino acid incorporation (C15M, M36V, M821, M88V, V114M) was optimized for E. coli expression, and directly synthesized as “gBlocks” from IDT. The gene was cloned into the pY71 vector using the same methods described above.

Cell-free protein synthesis (CFPS). The CD47 ECD mutants were expressed using the PANOx-SP (PEP, amino acids, nicotinamide adenine dinucleotide (NAD), oxalic acid, spermidine, and putrescine) CFPS system to produce disulfide-bonded proteins as described previously with some modifications, Lu et al. Proc. Natl. Acad. Sci. 201510533 (2015). First, the cell extract was pretreated with 0.5 mM iodoacetamide for 30 min at room temperature. The CFPS reaction mixture was then supplemented with 4 mM oxidized glutathione (GSSG, AppliChem) and 1 mM reduced glutathione (GSH) to stabilize the thiol/disulfide redox potential. Also, a disulfide bond isomerase, E. coli DsbC, was added. Finally, to improve protein solubility, the potassium glutamate concentration was reduced from the original 175 mM to 50 mM. The final reaction mixture includes: 10 mM magnesium glutamate, 10 mM ammonium glutamate, 50 mM potassium glutamate, 4 mM GSSG, 1 mM GSH, 100 μg/mL DsbC, 1.25 mM ATP, 1 mM each of GTP, UTP, and CTP, 34 μg/mL folinic acid, 170.6 μg/mL tRNA (Roche Molecular Biochemicals), 2 mM each of 18 amino acids (all but Met and Glu), 33.3 mM phosphoenolpyruvate (PEP, Roche Molecular Biochemicals), 0.33 mM NAD, 0.27 mM coenzyme A (CoA), 2.7 mM potassium oxalate, 1 mM putrescine, 1.5 mM spermidine, 2 mM methionine, 6 μM plasmid DNA that encodes the protein of interest under the T7 promoter, approximately 100-300 μg/mL purified T7 RNA polymerase, and 0.25 volumes of pretreated KC6 S30 extract (prepared from A19 met+, ΔtonA, ΔtnaA, ΔspeA, ΔendA, ΔsdaA, ΔsdaB, ΔgshA E. coli cells). All reagents were obtained from Sigma-Aldrich unless otherwise indicated. To quantify the protein yields, 2-10 μM L-[U-¹⁴C]-leucine (PerkinElmer) was also added. For global methionine replacement to incorporate nnAAs, methionine was left out of the CFPS reaction mixture, and either 2 mM azidohomoalanine (AHA, Medchem Source LLP), or 2 mM homopropargylglycine (HPG, Chiralix) was added instead.

The reactions were conducted at multiple scales: in 15-50 μL volumes in 2 mL Eppendorf tubes, 500 μL volumes in sealed 6-well culture plates, or 5 mL volumes in sealed 10 cm petri dishes. The reactions were incubated at either 30° C. or room temperature for 16 hours.

Analysis of synthesized protein concentration and solubility. Protein concentrations were determined by measuring the incorporated radioactivity using a Beckman LS6000 liquid scintillation counter. Total expressed protein concentrations were measured directly and soluble protein concentrations were measured after removing the insoluble fractions using centrifugation at 10,000×g for 15 min. Both samples (3-5 μL) were spotted and precipitated on Whatman filter paper before being submerged three times in 5% trichloroacetic acid (TCA) to remove unincorporated ¹⁴C-leucine.

The protein solubility is defined as the percent of the total amount of produced ¹⁴C-labeled protein that can be precipitated from the supernatant after centrifugation (soluble protein).

$$Protein solubility = \frac{amount of soluble produced protein}{total amount of produced protein} \times 100 \%$$

Purification of the CD47 ECD with N-terminal extension using immobilized metal affinity chromatography (IMAC). The CD47 ECD fusion proteins (His₆-EKseq-CD47 ECD) were expressed using CFPS as described above. The purification method first used Sephadex G-25 PD-10 Desalting Columns (GE Healthcare Life Sciences) to exchange the CFPS reaction macromolecules into 50 mM Tris-HCl pH 7.4, 25 mM Imidazole, 0.01% Tween-20. Next, the insolubles were removed using centrifugation at 10,000×g for 15 min before loading the soluble fraction (supernatant) onto the nickel-nitrilotriacetic acid (Ni-NTA) column (Qiagen), which was equilibrated with 50 mM Tris-HCl pH 7.4, 25 mM Imidazole, 0.01% Tween-20. The samples were washed with 7 column volumes (CVs) of the same buffer, and eluted with 5 CVs of 50 mM Tris-HCl pH 7.4, 250 mM imidazole, 0.01% Tween-20 while collecting 0.5 CV fractions. Then, the fractions containing the CD47 ECD fusion protein were pooled and concentrated using Amicon® Ultra Centrifugal Filters (10 kDa molecular weight cutoff) (Millipore).

Production of the CD47 ECD with pyroglutamate at the N-terminus. The purified CD47 ECD fusion proteins (His₆-EKseq-CD47 ECD) were first loaded onto Sephadex G-25 PD-10 Desalting Columns (GE Healthcare Life Sciences) to exchange the proteins into 50 mM Tris-HCl pH 7.4, 50 mM NaCl, 1 mM CaCl₂, 0.01% Tween-20. Next, the proteins were digested with human His-tagged enterokinase cleavage enzyme (Applied Biological Materials) at 4° C. for 20-23 hours. Then, the uncleaved fusion protein (His₆-EKseq-CD47 ECD), cleaved N-terminal tag (Hiss-EKseq), and His-tagged enterokinase were removed by Ni-NTA IMAC as described above. The mature CD47 ECD proteins that did not bind to the Ni-NTA resin (Qiagen) were collected from the flow through and wash fractions, and loaded onto the Sephadex G-25 PD-10 Desalting Columns (GE Healthcare Life Sciences) to exchange into 50 mM Tris-HCl, 0.01% Tween-20. Finally, the protein samples were concentrated using Amicon® Ultra Centrifugal Filters (10 kDa molecular weight cutoff) (Millipore), and treated with glutaminyl cyclase (QC, Novus biologicals) at room temperature for 1-3 hours to convert the N-terminal glutamine residue to pyroglutamate.

Pyroglutamate formation detection. The conversion efficiency of the glutamine residue on the CD47 ECD N-terminus to pyroglutamate was examined using the spectrophotometric continuous assay established by Schilling et al. Briefly, 50 μM of the (1Q)CD47 ECD protein was mixed with 150 μM NADH (Sigma), 7 mM α-ketoglutarate (Sigma), 15 U/mL glutamate dehydrogenase (GLDH, Sigma), and the QC enzyme in 50 mM Tris-HCl pH 7.4 buffer. The decrease of the NADH concentration, which reflects the pyroglutamate formation, was continuously detected by measuring absorbance at 340 nm using a SpectraMax® iD3 Mufti-Mode Microplate Reader (Molecular Devices).

HepBc VLP production. The HepBc mutants previously developed to load small molecules (HepBc HP 2ASVins SS1 SS8 79M (IG)₂IC-His₆ and HepBc HP 2ASVins SS1 SS8 80M (IG)₂IC-His₆) were used in this study. The HepBc proteins containing AHA instead of methionine were expressed using CFPS, and the VLPs displaying azide functional groups were produced as described previously (U.S. Patent Application No. 62/788,558). Empty VLPs were used for the assessment of the CD47 ECD attachment on the VLP surface, and Bodipy FL Cysteine (BDFL)-loaded VLPs were used for the macrophage avoidance assay.

Copper(I)-catalyzed azide-alkyne cycloaddition (“click” chemistry). For “click” reaction attachment, the CD47 ECD proteins were expressed with HPG (containing an alkyne functional group), and the HepBc mutant proteins were expressed with AHA (containing an azide functional group) instead of methionine using CFPS. The CD47 ECD proteins were conjugated to the HepBc VLP surface based on the previously developed protocol (Patel & Swartz Bioconjug. Chem. 22, 376-387 (2011)). Reactions were conducted in an anaerobic glovebox (Coy Laboratories) to preserve the reduced state of the tetrakis(acetonitrile)copper(I) hexafluorophosphate catalyst (Sigma Aldrich). 41.67 nM HepBc VLPs (monomer protein concentration was 10 μM) were combined with 1.5-20 μM CD47 ECD, 0.5 mM tris(triazolylmethyl) amine (TTMA), 0.01% Tween-20, and 2 mM tetrakis catalyst in 50 mM phosphate buffer pH 8 and allowed to react for 16 hours anaerobically.

Analysis of “click” chemistry bioconjugation using sodium dodecyl sulfate polyacrylamide gel electrophoresis (SDS-PAGE) and autoradiography. For the analysis of bioconjugation, non-radioactive CD47 ECD and radioactively-labeled HepBc VLPs were used, and the bioconjugated product size was analyzed by SDS-PAGE and autoradiography. NuPAGE Novex precast gels and reagents were purchased from Invitrogen. For reducing SDS-PAGE, samples were mixed with LDS running buffer and 50 mM dithiothreitol (DTT), and denatured for 10 min at 75° C. The samples were loaded onto a 12% (w/v) Bis-Tris precast gel with a separate lane for the Mark 12 molecular weight protein standard (Thermo Fisher Scientific), and electrophoresed in MES/SDS running buffer. SimplyBlue SafeStain (Thermo Fisher Scientific) was used to stain and fix the gels according to the manufacturer's recommendations. The gels were dried using a gel dryer model 583 (Bio-Rad), before exposure to a storage phosphor screen (Molecular Dynamics), which was subsequently scanned using a Typhoon Scanner (GE Healthcare) for radioautography. The disassembled and reduced HepBc monomer protein band and the conjugated product bands (HepBc monomer+CD47 ECD and two HepBc monomers+CD47 ECD) could be seen on the autoradiograph. Based on the density of control band and the conjugate bands, the conjugation efficiency was estimated by densitometry using ImageJ software.

CD47 ECD-functionalized VLP purification using size-exclusion chromatography (SEC). After the “click” reactions, the copper(I) catalyst and unconjugated ligands were removed by SEC. The CD47 ECD-functionalized HepBc VLPs were purified by applying 200 μL of the reaction solution to 2.2 mL Sepharose 6 Fast Flow resin (GE Healthcare), eluting 24×150 μL fractions. PBS containing 0.01% Tween-20 was used to equilibrate the SEC column and to elute the VLPs. The VLP peak fractions (typically between fractions 5 and 8) were detected by measuring fluorescent intensity of the loaded fluorescent dye BDFL. Finally, the VLP fractions were pooled and concentrated using Amicon® Ultra Centrifugal Filters (30 kDa molecular weight cutoff) (Millipore).

Macrophage avoidance assay. The ability of the displayed engineered mouse CD47 ECD mutant to avoid VLP phagocytosis was examined using mouse macrophage-like RAW 264.7 cells constitutively expressing H2B fused with miRFP670 (obtained from the Markus Covert Lab) to provide nuclear labeling. Two days before imaging, these RAW 264.7 cells were seeded at 7,500 cells/well in a glass-bottom, 96-well tissue culture plate that had been precoated with 10 μg/mL human fibronectin (FC010, Millipore). Then, one day before imaging, cells were stimulated with 100 ng/mL lipopolysaccharide (LPS, from E. coli, Serotype EH100(Ra), Enzo Life Sciences) and 20 ng/mL murine interferon gamma (IFNγ, PeproTech) in 100 μL of DMEM (Life Technologies) supplemented with 10% FBS (Omega Scientific), 2 mM L-glutamine (Life Technologies), and 1× penicillin/streptomycin (P/S, Life Technologies) for 20 hours to allow them to differentiate for enhanced phagocytosis. On the day of imaging, the cells were incubated with 0.05 nM (final concentration) BDFL-loaded VLPs at 37° C. for 2 hours to allow phagocytosis, followed by three cell washes with PBS containing 0.1% Tween-20 to remove free VLPs. Lastly, the media was changed into FluoroBrite DMEM (Life Technologies) supplemented with 1% FBS and 2 mM L-glutamine for imaging. A Nikon Eclipse Ti-E inverted microscope with an Andor Neo 5.5 sCMOS Camera was used for fluorescent imaging to detect the phagocytosed VLPs and the BDFL dye molecules in the RAW 264.7 cells. Far-red channel (645/30-nm excitation filter and 705/72-nm emission filter) and FITC channel (490/20-nm excitation filter and 525/36-nm emission filter) were used to detect the cell nuclei and the BDFL dyes, respectively.

### Results and Discussion

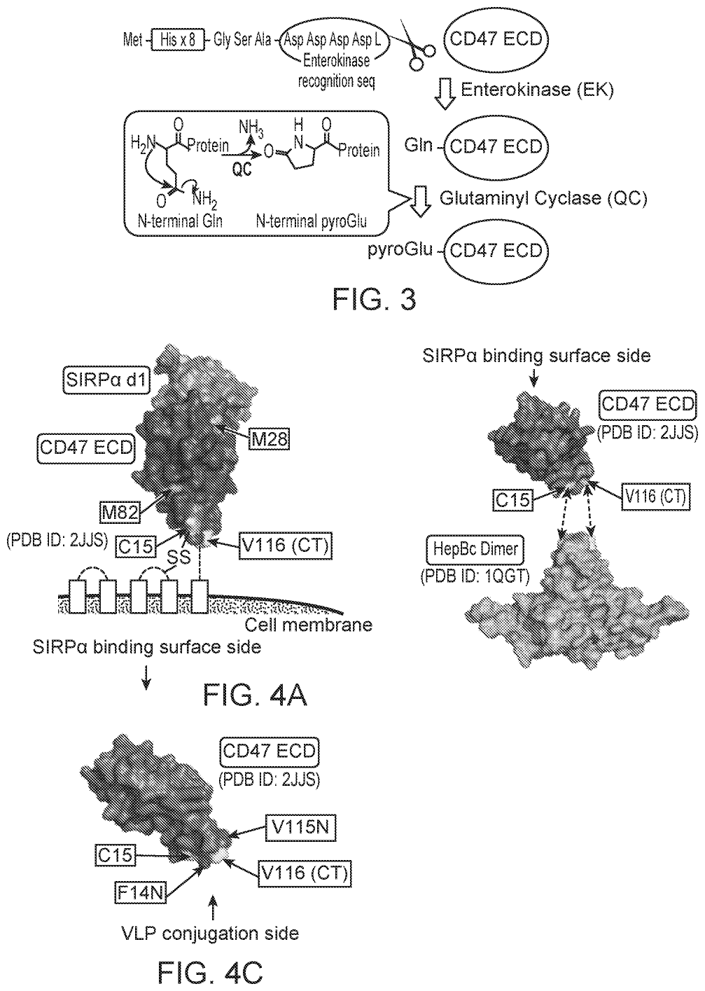

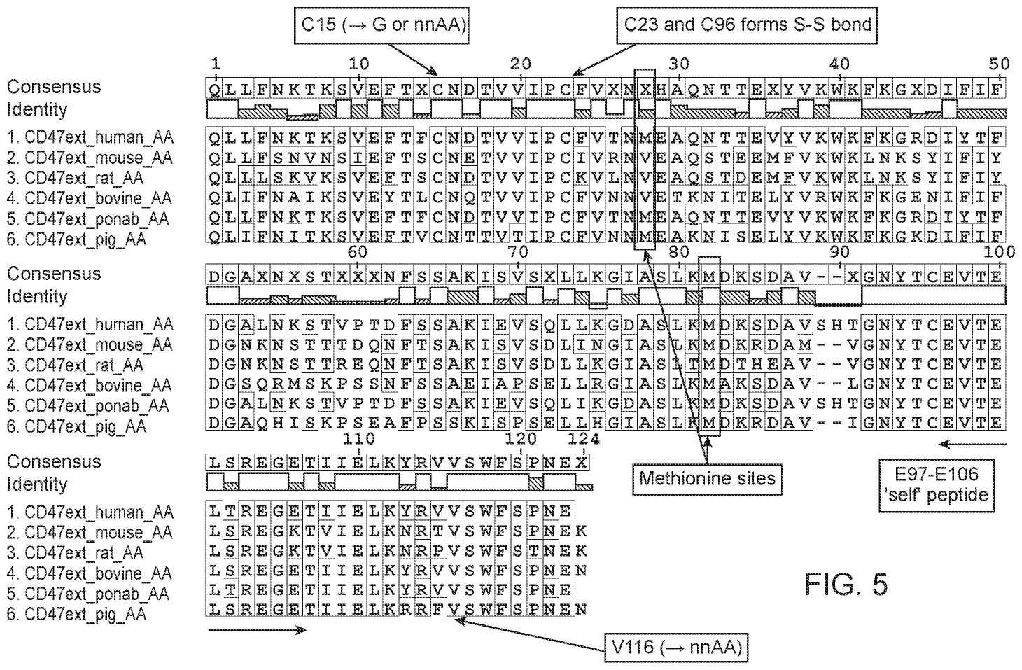

Cell-free expression of the human CD47 extracellular domain with an N-terminal extension. We first designed the human CD47 ECD with an N-terminal extension that allows production of the CD47 ECD with a glutamine N-terminus that can be converted into the authentic pyroglutamate terminus. This is important for SIRPα recognition. On the N-terminus, we introduced an octahistidine tag (His×8) for purification, followed by a short Gly Ser Ala linker and the enterokinase recognition sequence (Asp Asp Asp Asp Lys) (FIG. 3). After purifying this fusion protein (His₆-EKseq-hCD47 ECD), the extension is removed immediately after the recognition sequence using enterokinase. This exposes the glutamine residue at the N-terminus, which then can be converted to pyroglutamate using glutaminyl cyclase.

Next, we introduced several mutations into the human CD47 ECD to enable nnAA incorporation using CFPS and global methionine replacement. Methionine analogs are incorporated instead of methionine using endogenous methionyl-tRNA synthetases, and this enables “click” conjugation to the VLP surface. First, we replaced two natural methionine residues (M28 and M82), which are not at the recognition interface between CD47 and SIRPα (FIG. A). Based on the CD47 conserved sequence search, M28 was replaced with valine (FIG.). On the other hand, M82 was conserved in most of the available CD47 amino acid sequences, thus several different mutants were created (example: M82L and M821). Then, we introduced two nnAA incorporation sites (ATG codons) at C15 and V116, the double linkage sites that anchor the ECD to the cell membrane (FIG. A). We hypothesized that, using “click” conjugation, the double linkage can be replicated on the HepBc VLP surface with the other two nnAAs incorporated at the protruding spike tip of the HepBc dimer subunit to achieve the authentic CD47-SIRPα binding (FIG. B). In addition to these mutations, two hydrophobic residues on the surface of CD47 ECD that faces the cell membrane (or the VLP surface) were replaced with asparagine residues, the most hydrophilic non-charged amino acid, to improve protein solubility (F14N and V115N) (FIG. C).

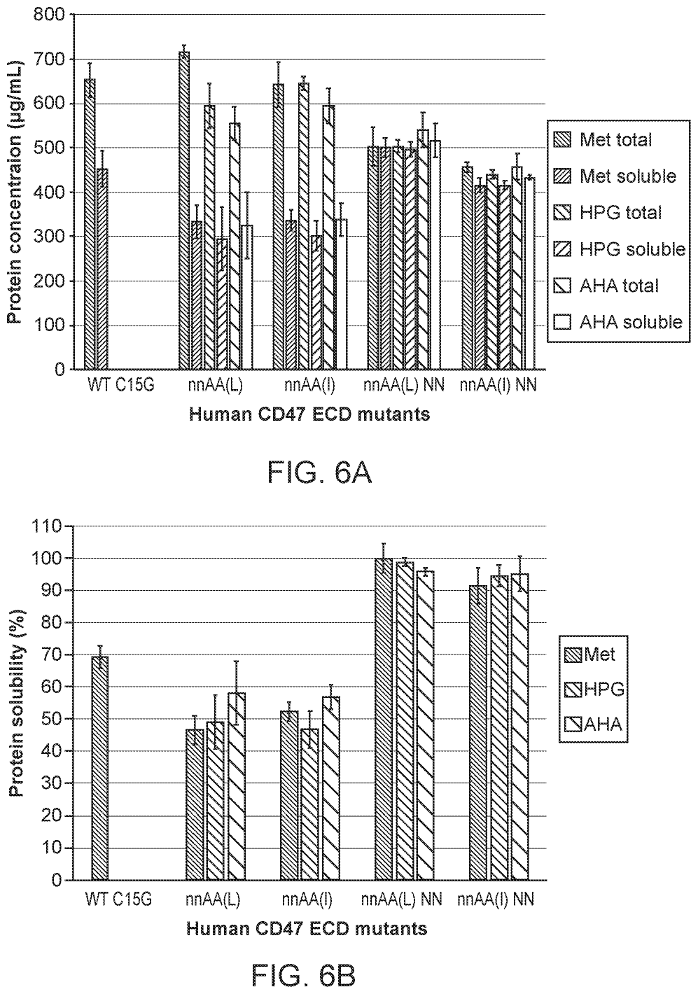

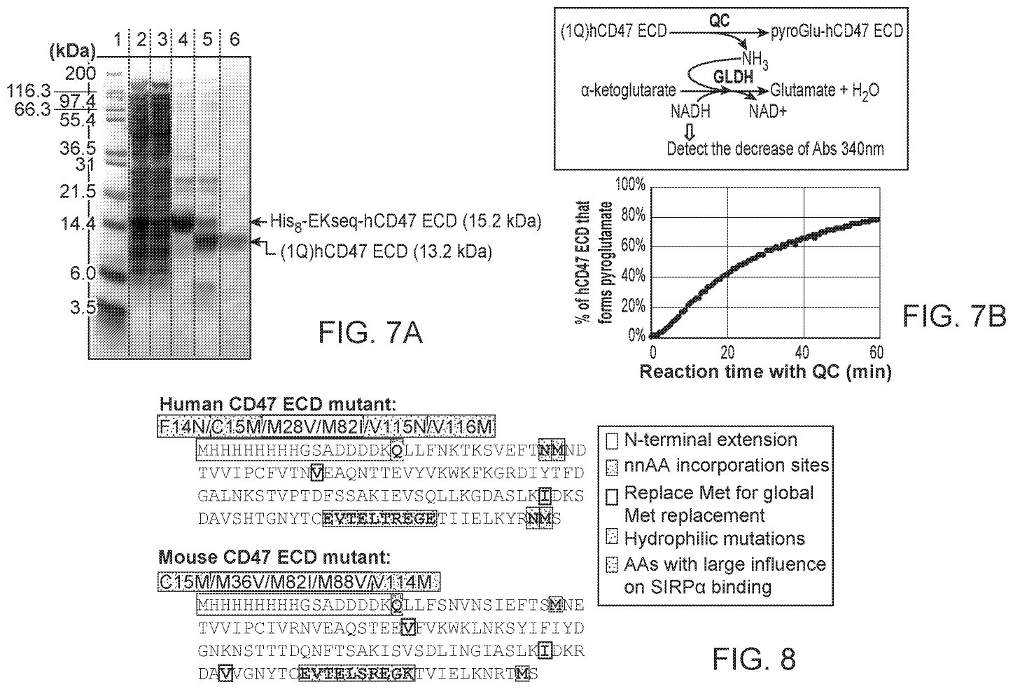

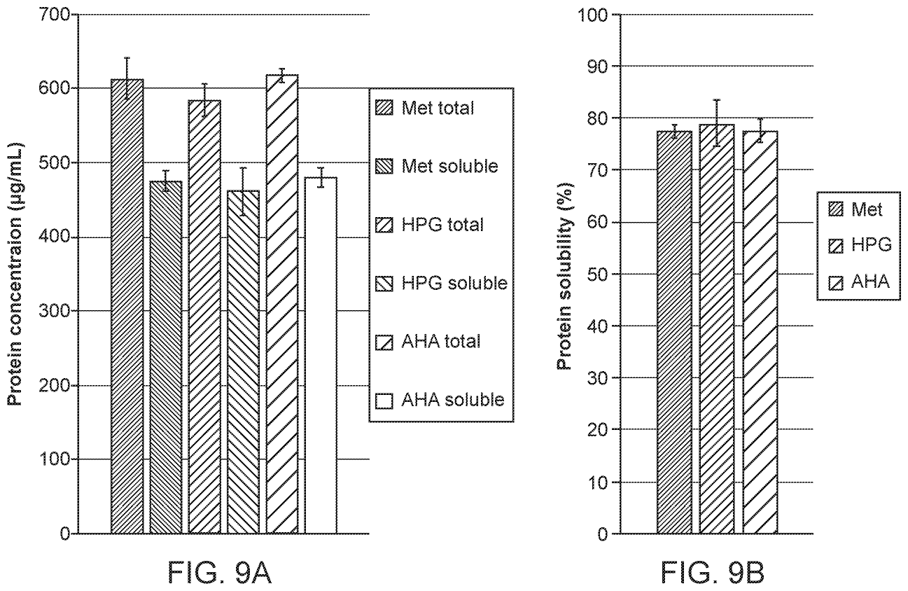

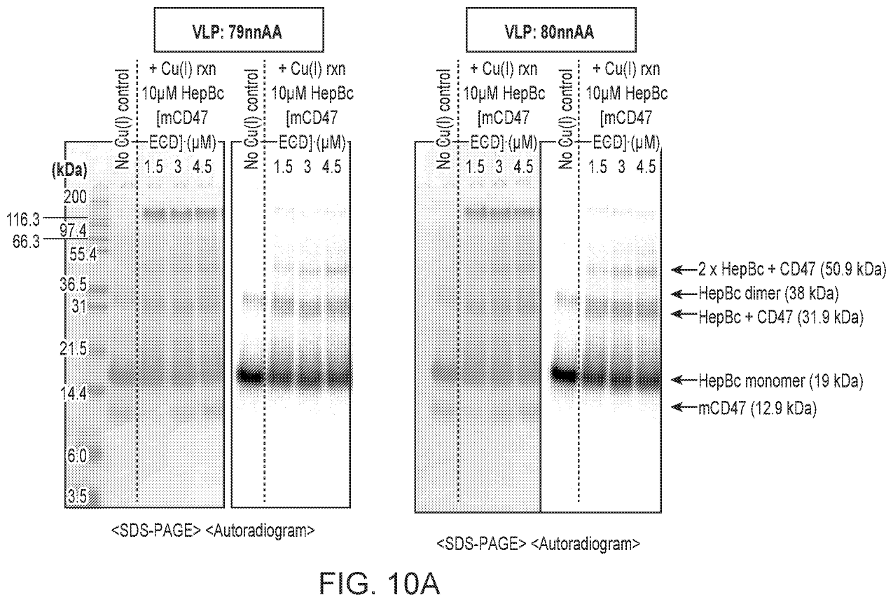

Using these mutants, we then tested the cell-free protein expression level and solubility using CFPS with methionine, with a nnAA containing an alkyne functional group (homopropargylglycine, HPG), or with a nnAA containing an azide functional group (azidohomoalanine, AHA) (FIG.). Compared to the mutant with only a C15G mutation (WT G15G), which was introduced to avoid protein aggregation due to incorrect disulfide bond formation, the soluble protein accumulation level decreased for mutants designed to avoid undesired nnAA incorporation (nnAA(L) and nnAA(I)). However, for the engineered CD47-ECDs with hydrophilic asparagine mutations (F14N+V115N, the mutants were designated as nnAA(L) NN and nnAA(I) NN); the soluble protein accumulation recovered to the WT C15G level and relative solubility increased from ˜70% to >90% (FIG. 6B). Those hydrophilic mutants were used for further study.

Purification of the human CD47 ECD. Using the engineered human CD47 ECD with the N-terminal extension and mutations described above, we next purified and cleaved the initial expression product to produce the human CD47 ECD with an N-terminal glutamine. The samples at each step of purification were evaluated by SDS-PAGE (FIG. A). The fusion protein was expressed using CFPS, and then purified using Ni-NTA IMAC, followed by enterokinase cleavage. During this protease reaction, we observed some nonspecific cleavage products—probably cleaved near 83D and 84K, because of the sequence similarity to the enterokinase recognition sequence (DDDDK). The level of this undesired cleavage changed based on the mutation at position 82. Since the nnAA(I) NN mutant suffered less nonspecific cleavage than the nnAA(L) NN mutant, the nnAA(I) NN mutant was selected for further study. Using Ni-NTA IMAC again, the enterokinase, cleaved N-terminal tag (Hiss-EKseq), and uncleaved fusion protein (Hiss-EKseq-hCD47 ECD) were all removed, leaving the human CD47 ECD nnAA(I) NN with an N-terminal glutamine ((1Q)hCD47 ECD). Then, we tested the conversion of glutamine to pyroglutamate by QC. Using the spectrophotometric continuous assay established by Schilling et al., up to 80% of pyroglutamate formation was indicated (FIG. B).

Production of the mouse CD47 ECD. Based on the experience with the human CD47 ECD, we next created the mouse version of CD47 ECD with the same mutations for future mouse studies, given the fact that CD47-SIRPα interaction is species-restricted. As shown in FIG., the original three methionine residues were replaced based on sequence conservation (M36V, M821, M88V) (FIG.). The hydrophilic mutations used for the human CD47 ECD (F14N and V115N) were not introduced to the mouse CD47 ECD, since it didn't have hydrophobic residues at these positions (corresponding amino acids are S14 and T113). Then, protein expression was tested using CFPS, and the mouse CD47 ECD with the N-terminal extension accumulated to the same concentration as the human version with about 80% protein solubility for CD47-ECD with met, HPG, and AHA incorporation (FIG.). The mouse CD47 ECD with N-terminal pyroglutamate was purified and produced using the same method described above for the human version.

Assessment of attachment of the mouse CD47 ECD to the HepBc VLP surface. We then tested bioconjugation of the purified mouse CD47 ECD to the HepBc VLP surface using “click” chemistry. A nnAA displaying an alkyne functional group (HPG) and a nnAA displaying an azide functional group (AHA) were introduced into the HepBc VLP and the mouse CD47 ECD, respectively. Previously, we tested several nnAA incorporation sites at the tip of the HepBc spike region shown in FIG. C. For the mouse CD47 ECD attachment, position 79 and 80 were tested; D78 was avoided to stabilize the HepBc helical structure as a capping residue, and L76 was not selected because it was not expected to achieve the designed double linkage with the CD47 ECD due to the relatively large distance between the two L76 residues in each dimer. The bioconjugation was performed with varied concentrations of the mouse CD47 ECD with radioactively-labeled HepBc VLPs (79nnAA VLPs and 80nnAA VLPs) in the presence of Cu(I), and then analyzed by reducing SDS-PAGE and autoradiography (FIG. A). For both types of the VLPs, in addition to the HepBc monomer bands, two bands indicating conjugation products were observed; one was for the CD47 ECD conjugated to one HepBc monomer (single linkage), and the other was for the CD47 ECD conjugated to two HepBc monomers (one dimer) (double linkage). FIG. B shows the number of the attached CD47 ECD per VLP based on gel densitometry. Since the band of HepBc dimers that were not fully reduced and the single linkage band were partially merged, the HepBc dimer band density was subtracted from the merged band density to calculate the single linkage band density. Although we had some single linkage conjugates, these results suggested that >10 mouse CD47 ECDs were conjugated to the HepBc VLP surface by double linkage, similar to their authentic anchoring to the cell membrane. Since 80nnAA VLPs showed a slightly higher ratio of the double linkage than the single linkage compared to 79nnAA VLPs, 80nnAA VLPs were used for further study.

Surface modification with the CD47 ECD reduces VLP uptake by macrophage-like cells. Based on the bioconjugation analysis, it was expected that the majority of attached mouse CD47 ECDs were displaying the SIRPα binding side on the VLPs in the authentic orientation. Therefore, we tested their function to avoid phagocytosis using mouse macrophage-like RAW 264.7 cells. RAW 264.7 cells were first stimulated with LPS and IFNγ to allow differentiation for more active phagocytosis, and then were incubated for 2 hours at 37° C. with the fluorescent dye BDFL-loaded VLPs that were previously produced with or without mouse CD47 ECD on the surface. Then, the media was removed and the cells were washed three times, followed by imaging under a fluorescent microscope. As shown in FIG., uptake of the mouse CD47 ECD-functionalized and BDFL-loaded VLPs by the stimulated RAW 264.7 cells was significantly reduced relative to the same VLPs without the CD47 ECD. This indicates that the engineered CD47 ECD retained its functionality to inhibit phagocytosis after being attached to the VLPs.

In this work, we have engineered the CD47 ECD for bioconjugation to the HepBc VLP and successfully demonstrated its functionality in avoiding phagocytic engulfment by macrophage-like cells. Particularly, our design to replicate the double linkage, which endogenously orients the CD47 ECD relative to the cell membrane surface, can provide authentic interaction with SIRPα and avoid immunogenicity. The CD47 ECD can also benefit other types of NPs that display pairs of reactive functional groups on their surface in appropriate proximity for “click” bioconjugation of the CD47 ECD.

The CD47 ECD functionality can be combined with a specific targeting functionality for effective targeted delivery using NPs. Examples of targeting ligands are single-chain variable fragments (scFvs) and DNA aptamers recognizing a specific cell surface marker of a targeted cells. One-pot “click” conjugation methods are advantageous to easily make NPs with different densities of each ligand by changing the relative ligand concentrations in the reaction.

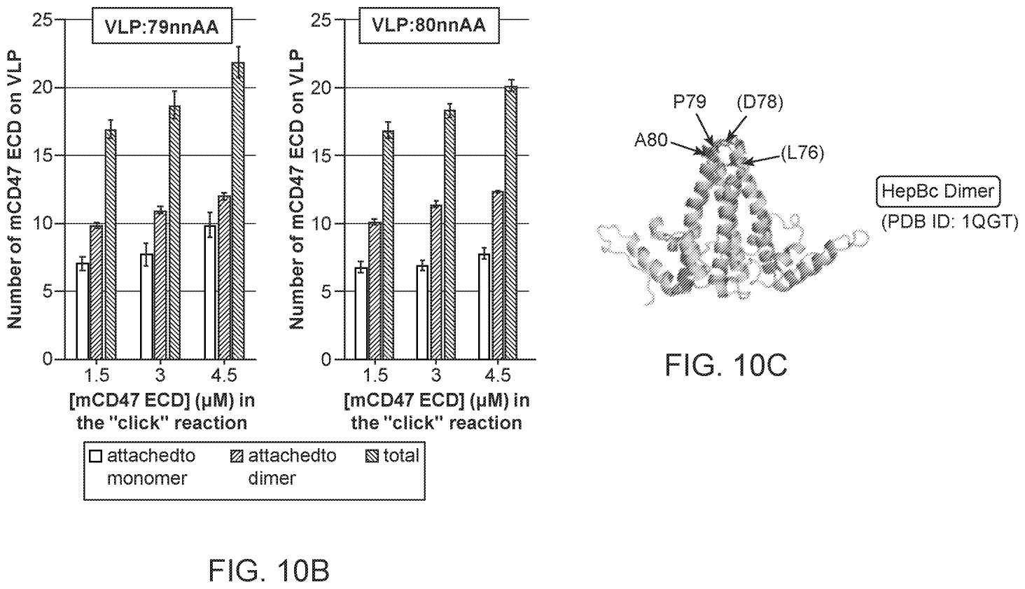

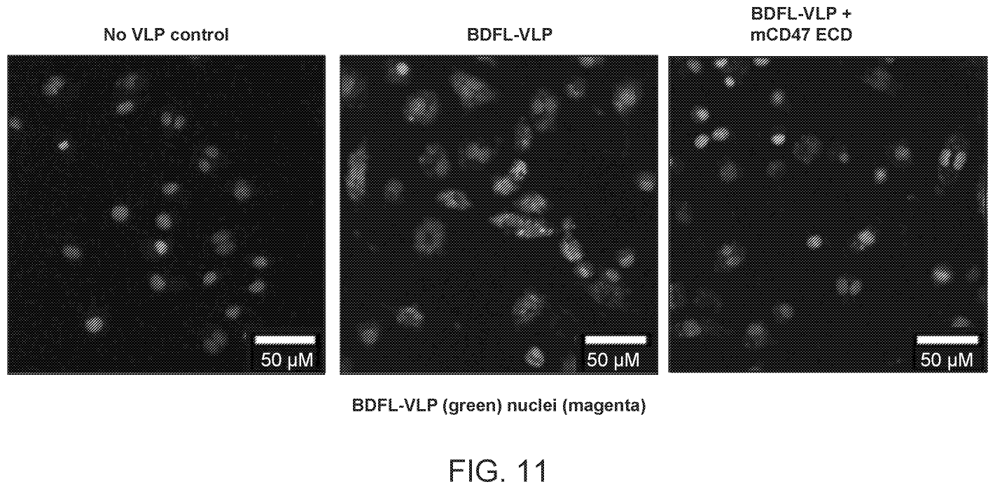

A benefit is that the relatively small number of attached CD47-ECDs on the surface of the NPs (FIG. 10B) still leaves about 100 surface spikes available for the subsequent or simultaneous attachment of cell targeting agents. Thus, while phagocytic clearance will be substantially reduced, the NP uptake by the targeted cells will not be lowered.

The following tables provide the protein and nucleotide sequences used in the examples. The non-natural amino acid (nnAA) incorporation sites are indicated as “Z” in the protein primary sequence, and the mutation sites to replace original methionine residues are shown; and asparagine hydrophilic mutation sites. The regions with large influence on SIRPα binding are underlined and the N-terminal extension is double underlined.

TABLE 1

| Mutants | Protein sequence | Gene sequence |
|---|---|---|
| His₈-EKseq- | (SEQ ID NO: 3) | (SEQ ID NO: 4) |
| hCD47 ECD | MHHHHHHHHGSADDDDKQLLFNKTKSVEFTF | ATGCACCATCATCATCACCACCATCACGGCA |
| WT C15G | GNDTVVIPCFVTNMEAQNTTEVYVKWKFKGR | GCGCGGATGACGATGACAAACAGCTGCTGTT |
| | DIYTFDGALNKSTVPTDESSAKIEVSQLLKG | TAACAAAACCAAAAGCGTGGAATTTACCTTT |
| | DASLKMDKSDAVSHTGNYTCEVTELTREGET | GGCAACGATACCGTGGTGATTCCGTGCTTTG |
| | IIELKYRVVS** | TGACCAACATGGAAGCGCAGAACACCACCGA |
| | | AGTGTATGTGAAATGGAAATTTAAAGGCCGC |
| | | GATATTTATACCTTTGATGGCGCGCTGAACA |
| | | AAAGCACCGTGCCGACCGATTTTAGCAGCGC |
| | | GAAAATTGAAGTGAGCCAGCTGCTGAAAGGC |
| | | GATGCGAGCCTGAAAATGGATAAAAGCGATG |
| | | CGGTGAGCCATACCGGCAACTATACCTGCGA |
| | | AGTGACCGAACTGACCCGCGAAGGCGAAACC |
| | | ATTATTGAACTGAAATATCGCGTGGTGAGCT |
| | | AATAA |
| His₈-EKseq- | (SEQ ID NO: 5) | (SEQ ID NO: 6) |
| hCD47 ECD | ZHHHHHHHHGSADDDDKQLLFNKTKSVEFTF | ATCCACCATCATCATCACCACCATCACCCCA |
| nnAA(L) | ZNDTVVIPCFVTNVEAQNTTEVYVKWKFKGR | GCGCGGATGACGATGACAAACAGCTGCTGTT |
| | DIYTFDGALNKSTVPTDFSSAKIEVSQLLKG | TAACAAAACCAAAAGCGTGGAATTTACCTTT |
| | DASLKLDKSDAVSHTGNYTCEVTELTREGET | ATGAACGATACCGTGGTGATTCCGTGCTTTG |
| | IIELKYRVZS** | TGACCAACGTGGAAGCGCAGAACACCACCGA |
| | | AGTGTATGTGAAATGGAAATTTAAAGGCCCC |
| | | GATATTTATACCTTTGATGGCGCGCTGAACA |
| | | AAAGCACCGTGCCGACCGATTTTAGCAGCGC |
| | | GAAAATTGAAGTGAGCCAGCTGCTGAAAGGC |
| | | GATGCGAGCCTGAAACTGGATAAAAGCGATG |
| | | CGGTGAGCCATACCCCCAACTATACCTGCGA |
| | | AGTGACCGAACTGACCCGCGAAGGCGAAACC |
| | | ATTATTGAACTGAAATATCGCGTGATGAGCT |
| | | AATAA |
| His₈-EKseq- | (SEQ ID NO: 7) | (SEQ ID NO: 8) |
| hCD47 ECD | ZHHHHHHHHGSADDDDKQLLFNKTKSVEFTF | ATGCACCATCATCATCACCACCATCACGGCA |
| nnAA(I) | ZNDTVVIPCFVTNVEAQNTTEVYVKNKFKGR | GCGCGGATGACGATGACAAACAGCTGCTGTT |
| | DIYTFDGALNKSTVPTDFSSAKIEVSQLLKG | TAAAAAACCAAAAGCGTGGAATTTACCTTT |
| | DASLKLDKSDAVSHTGNYTCEVTELTRECET | ATGAACGATACCGTGGTGATTCCGTGCTTTG |
| | IIELKYRVZS** | TGACCAACGTGGAAGCGCAGAACACCACCGA |
| | | AGTGTATGTGAAATGGAAATTTAAAGGCCGC |
| | | GATATTTATACCTTTGATGGCGCGCTGAACA |
| | | AAAGCACCGTGCCGACCGATTTTAGCAGCGC |
| | | GAAAATTGAAGTGAGCCAGCTGCTGAAAGGC |
| | | GATGCGAGCCTGAAAATTGATAAAAGCGATG |
| | | CGGTGAGCCATACCGGCAACTATACCTGCGA |
| | | ACTGACCGAACTGACCCGCGAAGGCGAAACC |
| | | ATTATTGAACTGAAATATCGCGTGATGAGCT |
| | | AATAA |
| His₈-EKseq- | (SEQ ID NO: 9) | (SEQ ID NO: 10) |
| hCD47 ECD | ZHHHHHHHHGSADDDDKQLLFNKTKSVEFTN | ATGCACCATCATCATCACCACCATCACGGA |
| nnAA(L)NN | SNDTVVIPCFVTNVEAQNTTEVYVKWKFKGR | GCGCGGATGACGATGACAAACAGCTGCTGTT |
| | DIYTFDGALNKSTVPTDFSSAKIEVSQLLKG | TAACAAAACCAAAAGCGTGGAATTTACCAAC |
| | DASLKLDKSDAVSHTGNYTCEVTELTREGET | ATGAACGATACCGTGGTGATTCCGTGCTTTG |
| | IIELKYRNZS** | TGACCAACGTGGAAGCGCAGAACACCACCGA |
| | | AGTGTATGTGAAATGGAAATTTAAAGGCCGC |
| | | GATATTTATACCTTTGATGGCGCGCTGAACA |
| | | AAAGCACCGTGCCGACCGATTTTAGCAGCGC |
| | | GAAAATTGAAGTGAGCCAGCTGCTGAAAGGC |
| | | GATGCGAGCCTGAAACTGGATAAAAGCGATG |
| | | CGGTGAGCCATACCGGCAACTATACCTGCGA |
| | | AGTGACCGAACTGACCCGCGAAGGCGAAACC |
| | | ATTATTGAACTGAAATATCGaAACATGAGCT |
| | | AATAA |
| His₈-EKseq- | (SEQ ID NO: 11) | (SEQ ID NO: 12) |
| hCD47 ECD | ZHHHHHHHHGSADDDDKQLLFNKTKSVEFTN | ATGCACCATCATCATCACCACCATCACGGCA |
| nnAA(I)NN | ZNDTVVIPCFVTNVEAQNTTEVYVKWKFKGR | GCGCGGATGACGATGACAAACAGCTGCTGTT |
| | DIYTFDGALNKSTVPTDFSSAKIEVSQLLKG | TAACAAAACCAAAAGCGTGGAATTTACCAAC |
| | DASLKIDKSDAVSHTGNYTCEVTELTREGET | ATGAACGATACCGTGGTGATTCCGTGCTTTG |
| | IIELKYRNZS** | TGACCAACGTGGAAGCGCAGAACACCACCGA |
| | | AGTGTATGTGAAATGGAAATTTAAAGGCCGC |
| | | GATATTTATACCTTTGATGGCGCGCTGAACA |
| | | AAAGCACCGTGCCGACCGATTTTAGaAGCGC |
| | | GAAAATTGAAGTGAGCCAGCTGCTGAAAGGC |
| | | GATGCGAGCCTGAAAATTGATAAAAGCGATG |
| | | CGGTGAGCCATACCGGCAACTATACCTGCGA |
| | | AGTGACCGAACTGACCCGCGAAGGCGAAACC |
| | | ATTATTGAACTGAAATATCGCAACATGAGCT |
| | | AATAA |
| His₈-EKseq- | (SEQ ID NO: 13) | (SEQ ID NO: 14) |
| mCD47 | ZHHHHHHHHGSADDDDKQLLFSNVNSIEFTS | ATGCACCATCATCATCACCACCATCACGGCA |
| ECD | ZNETVVIPCIVRNVEAQSTEEVFVKWKLNKS | GCGCGGATGACGATGACAAACAGCTGCTGTT |
| nnAA(I) | YIFIYDGNKNSTTTDQNFTSAKISVSDLING | TAGCAACGTGAAAGCATTGAATTTACCAGC |
| | IASLKIDKRDAVVGNYTCEVTELSREGKTVI | ATGAACGAACCGTGGTGATTCCGTGCATTG |
| | ELKNRTZS** | TGCGCAACGTGGAAGCGCAGAGCACCGAAGA |
| | | AGTGTTTGTGAAATGGAAACTGAACAAAAGC |
| | | TACATCTTCATTTATGATGGCAACAAAAACA |
| | | GCACCACCACCGATCAGAACTTTACCAGCGC |
| | | GAAAATTAGCGTGAGCGATCTGATTAACGGC |
| | | ATTGCGAGCCTGAAAATTGATAAACGCGATG |
| | | CGGTGGTGGGCAACTATACCTGCGAAGTGAC |
| | | CGAACTGAGCCGCGAAGGCAAAACCGTGATT |
| | | GAACTGAAAAACCGCACCATGAGCTAATAA |

What is claimed is:

1. An engineered CD47 extracellular domain polypeptide (ECD), comprising:
(i) an N-terminal extension comprising a cleavage recognition site immediately adjacent to residue Q1 with reference to numbering of SEQ ID NO:1 or SEQ ID NO:2; and
(ii) non-natural amino acids (nnAA) at two sites of the CD47-ECD for attachment to a surface.

2. The engineered CD47-ECD of claim 1, wherein the nnAA are present at residues 15 and 116.

3. The engineered CD47-ECD of claim 1 or claim 2, wherein the nnAA are introduced by orthogonal translation components.

4. The engineered CD47-ECD of claim 1 or claim 2, wherein the nnAA are introduced by methionine replacement.

5. The engineered CD47-ECD of claim 4, comprising amino acid substitutions at residues M28 and M82 with amino acids other than methionine.

6. The engineered CD47-ECD of claim 5 wherein the amino acids other than methionine are selected from L, I, V, and F.

7. The engineered CD47-ECD of claim 6, wherein the amino acid substitutions are M28V; and M82L/I.

8. The engineered CD47-ECD of any of claims 1-7, wherein the nnAA comprise an alkyne or azide functional group.

9. The engineered CD47-ECD of claim 8, wherein the nnAA is selected from homopropargylglycine or azidohomoalanine.

10. The engineered CD47-ECD of any of claims 1-9, wherein the N-terminal extension comprises a cleavage recognition sequence for enterokinase.

11. The engineered CD47-ECD of any of claims 1-9, wherein the N-terminal extension comprises a cleavage recognition sequence for Factor Xa.

12. The engineered CD47-ECD of any of claims 1-11, wherein the N-terminal extension comprises a polypeptide tag for purification.

13. The engineered CD47-ECD of any of claims 1-12, wherein the N-terminal extension is cleaved, thereby exposing an N-terminal glutamine that is converted to pyroglutamate.

14. The engineered CD47-ECD of any of claims 1-13, comprising at least one amino acid substitution to replace hydrophobic residues on the surface of CD47 ECD that in the native protein faces the cell membrane with hydrophilic residues.

15. The engineered CD47-ECD of claim 14, wherein residues F14 and V115 are replaced with hydrophilic, non-charged amino acids.

16. The engineered CD47-ECD of claim 15, comprising one or both amino acid substitutions F14N and V115N.

17. The engineered CD47-ECD of any of claims 1-16, produced by cell free protein synthesis.

18. An article conjugated on its surface to an engineered CD47-ECD according to any of claims 1-17, through reaction to the nnAA.

19. The article of claim 18, wherein the reaction is copper(I)-catalyzed azide-alkyne cycloaddition.

20. The article of claim 18 or 19, wherein the article is a nanoparticle.

21. The article of claim 20, wherein the nanoparticle is a virus like particle.

22. The article of claim 21, wherein the virus like particle comprises hepatitis B core protein conjugated to the CD47-ECD.

23. The article of any of claims 18-22, comprising a therapeutic agent.

24. The article of any of claims 18-23, wherein phagocytic clearance of the article is reduced by at least 10% relative to an article in the absence of the CD47-ECD.

25. A method of coating an article with an engineered CD47-ECD of any of claims 1-17, the method comprising reacting reactive alkyne or azide groups on the surface of the article with nnAA present in the CD47-ECD.

26. The method of claim 25, wherein the reactive alkyne or azide groups are spaced from about 5 to about 15 Å apart.

27. The method of claim 25, wherein an array of reactive alkyne or azide groups is provided on the surface, providing a plurality of reactants.

28. The method of claim 27 wherein an array of reactive alkyne or azide groups is provided on the surface, nanoparticles displaying the conjugate alkyne or azide groups are first attached, and an engineered CD47-ECD is then attached to the nanoparticles.


SEQUENCE LISTING (14 sequences)

SEQ ID NO: 1 - protein, 117 aa, Human
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVS

SEQ ID NO: 2 - protein, 114 aa, Mouse
QLLFSNVNSIEFTSCNETVVIPCIVRNVEAQSTEEMFVKWKLNKSYIFIYDGNKNSTTTDQNFTSAKISVSDLINGIASLKMDKRDAMVGNYTCEVTELSREGKTVIELKNRTS

SEQ ID NO: 3 - protein, 134 aa, Artificial Sequence
  Synthetic Sequence
MHHHHHHHHGSADDDDKQLLFNKTKSVEFTFGNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVS

SEQ ID NO: 4 - DNA, 408 bp, Artificial Sequence
  Synthetic Sequence
atgcaccatcatcatcaccaccatcacggcagcgcggatgacgatgacaaacagctgctgtttaacaaaaccaaaagcgtggaatttacctttggcaacgataccgtggtgattccgtgctttgtgaccaacatggaagcgcagaacaccaccgaagtgtatgtgaaatggaaatttaaaggccgcgatatttatacctttgatggcgcgctgaacaaaagcaccgtgccgaccgattttagcagcgcgaaaattgaagtgagccagctgctgaaaggcgatgcgagcctgaaaatggataaaagcgatgcggtgagccataccggcaactatacctgcgaagtgaccgaactgacccgcgaaggcgaaaccattattgaactgaaatatcgcgtggtgagctaataa

SEQ ID NO: 5 - protein, 134 aa, Artificial Sequence
  Synthetic Sequence
ZHHHHHHHHGSADDDDKQLLFNKTKSVEFTFZNDTVVIPCFVTNVEAQNTTEVYVKWKFKGRDIYTFDGALNKSTVPTDFSSAKIEVSQLLKGDASLKLDKSDAVSHTGNYTCEVTELTREGETIIELKYRVZS

SEQ ID NO: 6 - DNA, 408 bp, Artificial Sequence
  Synthetic Sequence
atgcaccatcatcatcaccaccatcacggcagcgcggatgacgatgacaaacagctgctgtttaacaaaaccaaaagcgtggaatttacctttatgaacgataccgtggtgattccgtgctttgtgaccaacgtggaagcgcagaacaccaccgaagtgtatgtgaaatggaaatttaaaggccgcgatatttatacctttgatggcgcgctgaacaaaagcaccgtgccgaccgattttagcagcgcgaaaattgaagtgagccagctgctgaaaggcgatgcgagcctgaaactggataaaagcgatgcggtgagccataccggcaactatacctgcgaagtgaccgaactgacccgcgaaggcgaaaccattattgaactgaaatatcgcgtgatgagctaataa

SEQ ID NO: 7 - protein, 134 aa, Artificial Sequence
  Synthetic Sequence
ZHHHHHHHHGSADDDDKQLLFNKTKSVEFTFZNDTVVIPCFVTNVEAQNTTEVYVKWKFKGRDIYTFDGALNKSTVPTDFSSAKIEVSQLLKGDASLKIDKSDAVSHTGNYTCEVTELTREGETIIELKYRVZS

SEQ ID NO: 8 - DNA, 408 bp, Artificial Sequence
  Synthetic Sequence
atgcaccatcatcatcaccaccatcacggcagcgcggatgacgatgacaaacagctgctgtttaacaaaaccaaaagcgtggaatttacctttatgaacgataccgtggtgattccgtgctttgtgaccaacgtggaagcgcagaacaccaccgaagtgtatgtgaaatggaaatttaaaggccgcgatatttatacctttgatggcgcgctgaacaaaagcaccgtgccgaccgattttagcagcgcgaaaattgaagtgagccagctgctgaaaggcgatgcgagcctgaaaattgataaaagcgatgcggtgagccataccggcaactatacctgcgaagtgaccgaactgacccgcgaaggcgaaaccattattgaactgaaatatcgcgtgatgagctaataa

SEQ ID NO: 9 - protein, 134 aa, Artificial Sequence
  Synthetic Sequence
ZHHHHHHHHGSADDDDKQLLFNKTKSVEFTNZNDTVVIPCFVTNVEAQNTTEVYVKWKFKGRDIYTFDGALNKSTVPTDFSSAKIEVSQLLKGDASLKLDKSDAVSHTGNYTCEVTELTREGETIIELKYRNZS

SEQ ID NO: 10 - DNA, 408 bp, Artificial Sequence
  Synthetic Sequence
atgcaccatcatcatcaccaccatcacggcagcgcggatgacgatgacaaacagctgctgtttaacaaaaccaaaagcgtggaatttaccaacatgaacgataccgtggtgattccgtgctttgtgaccaacgtggaagcgcagaacaccaccgaagtgtatgtgaaatggaaatttaaaggccgcgatatttatacctttgatggcgcgctgaacaaaagcaccgtgccgaccgattttagcagcgcgaaaattgaagtgagccagctgctgaaaggcgatgcgagcctgaaactggataaaagcgatgcggtgagccataccggcaactatacctgcgaagtgaccgaactgacccgcgaaggcgaaaccattattgaactgaaatatcgcaacatgagctaataa

SEQ ID NO: 11 - protein, 134 aa, Artificial Sequence
  Synthetic Sequence
ZHHHHHHHHGSADDDDKQLLFNKTKSVEFTNZNDTVVIPCFVTNVEAQNTTEVYVKWKFKGRDIYTFDGALNKSTVPTDFSSAKIEVSQLLKGDASLKIDKSDAVSHTGNYTCEVTELTREGETIIELKYRNZS

SEQ ID NO: 12 - DNA, 408 bp, Artificial Sequence
  Synthetic Sequence
atgcaccatcatcatcaccaccatcacggcagcgcggatgacgatgacaaacagctgctgtttaacaaaaccaaaagcgtggaatttaccaacatgaacgataccgtggtgattccgtgctttgtgaccaacgtggaagcgcagaacaccaccgaagtgtatgtgaaatggaaatttaaaggccgcgatatttatacctttgatggcgcgctgaacaaaagcaccgtgccgaccgattttagcagcgcgaaaattgaagtgagccagctgctgaaaggcgatgcgagcctgaaaattgataaaagcgatgcggtgagccataccggcaactatacctgcgaagtgaccgaactgacccgcgaaggcgaaaccattattgaactgaaatatcgcaacatgagctaataa

SEQ ID NO: 13 - protein, 132 aa, Artificial Sequence
  Synthetic Sequence
ZHHHHHHHHGSADDDDKQLLFSNVNSIEFTSZNETVVIPCIVRNVEAQSTEEVFVKWKLNKSYIFIYDGNKNSTTTDQNFTSAKISVSDLINGIASLKIDKRDAVVGNYTCEVTELSREGKTVIELKNRTZS

SEQ ID NO: 14 - DNA, 402 bp, Artificial Sequence
  Synthetic Sequence
atgcaccatcatcatcaccaccatcacggcagcgcggatgacgatgacaaacagctgctgtttagcaacgtgaacagcattgaatttaccagcatgaacgaaaccgtggtgattccgtgcattgtgcgcaacgtggaagcgcagagcaccgaagaagtgtttgtgaaatggaaactgaacaaaagctacatcttcatttatgatggcaacaaaaacagcaccaccaccgatcagaactttaccagcgcgaaaattagcgtgagcgatctgattaacggcattgcgagcctgaaaattgataaacgcgatgcggtggtgggcaactatacctgcgaagtgaccgaactgagccgcgaaggcaaaaccgtgattgaactgaaaaaccgcaccatgagctaataa